(19) United States
(12) Patent Application Publication
(10) Pub. No.: US 2010/0260853 A1
(43) Pub. Date: Oct. 14, 2010

(54) COMPOSITIONS FOR PULMONARY DELIVERY

(76) Inventors: Amrik Basran, Cambridgeshire (GB); Neil D. Brewis, Cambridgeshire (GB); Catherine A. Sparks, Cambridgeshire (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/747,203

(22) PCT Filed: Dec. 11, 2008
(86) PCT No.: PCT/EP2008/067295

§ 371 Date: Jun. 10, 2010

(30) Foreign Application Priority Data
Dec. 13, 2007 (GB) 0724331.4
May 22, 2008 (BD) 127/2008
May 22, 2008 (BD) 128/2008
May 22, 2008 (BD) 129/2008
May 22, 2008 (BD) 130/2008
Jun. 3, 2008 (GB) PCT/GB2008/050399
Jun. 3, 2008 (GB) PCT/GB2008/050400
Jun. 3, 2008 (GB) PCT/GB2008/050403

(51) Int. Cl. A61K 9/14; A61K 39/395; A61K 38/16; A61P 11/00

(52) U.S. Cl. 424/489; 424/130.1; 514/21.2; 424/172.1

(57) ABSTRACT

The present invention relates to methods of direct pulmonary delivery of polypeptides e.g. of domain antibodies, and to particular polypeptide compositions suitable for direct pulmonary delivery. The invention also relates to use of such compositions in medicine, e.g. for the treatment and diagnosis of lung disease, for example for treating Chronic Obstructive Pulmonary Disease (COPD) and asthma.

The present invention relates to methods of direct pulmonary delivery of polypeptides e.g. of domain antibodies, and to particular polypeptide compositions suitable for direct pulmonary delivery. The invention also relates to use of such compositions in medicine, e.g. for the treatment and diagnosis of lung disease, for example for treating Chronic Obstructive Pulmonary Disease (COPD) and asthma.

### BACKGROUND OF THE INVENTION

Therapeutic or diagnostic agents are often are unable to penetrate tissues or organs to produces a desired therapeutic or diagnostic effect at a particular desired location

Hence a need exists for methods for directly administering such therapeutic or diagnostic agents directly to tissues or organs, for example directly to pulmonary tissue, and to produce a long therapeutic window for the agent.

A need also exists for particular compositions comprising such therapeutic or diagnostic agents, which are especially suitable for direct administration to particular tissues or organs, e.g. for compositions comprising therapeutic or diagnostic agents which are especially suitable for direct administration to the lung. Such compositions can be useful for treatment, diagnosis or prevention of disease e.g. for treatment, diagnosis or prevention of a respiratory disease, wherein the agent is formulated for direct local administration to pulmonary tissue. An example of such an agent is a domain antibody (dAb).

It has been found that domain antibodies (dAbs) that bind a target in pulmonary tissue can be useful in manufacture of a composition for treatment or prevention of a respiratory disease, wherein the composition is suitable for local administration to pulmonary tissue. In one embodiment, up to about 10 mg/kg of a dAb that binds a target in pulmonary tissue can be used. In another embodiment, the target in pulmonary tissue mediates lung inflammation or a pulmonary disease (see the disclosure in WO 2007049017 the contents of which are incorporated herein by reference)

One target of interest is the Tumor Necrosis Factor (TNF-α) pathway which is believed to be involved in the pathogenesis of lung diseases such as COPD and asthma.

Certain domain Antibodies (dAbs) have been generated that inhibit TNFR1 and these have been described (for example in WO 2007049017 and in WO 06038027) and they can be effective in treating lung inflammation or respiratory diseases, such as chronic obstructive pulmonary disease (COPD)

TNF-α has a broad spectrum of inflammatory effects relevant to COPD resulting in activation of neutrophils, monocytes, macrophages epithelium, mucus secretion and destruction of lung parenchyma through the release of proteinases (Barnes P J, et al., Chronic obstructive pulmonary disease: molecular and cellular mechanisms. Eur Respir J. 2003 October; 22(4):672-88).

Published studies have shown that TNF-α is present at elevated concentrations in the induced sputum of COPD patients compared to normal smokers and asthma patients (Keatings V M, et al., Differences in interleukin-8 and tumor necrosis factor-alpha in induced sputum from patients with chronic obstructive pulmonary disease or asthma. Am J Respir Crit. Care Med. 1996 February; 153(2):530-4).

In addition, increased expression of TNF-α may be temporally associated with exacerbation episodes in COPD patients (Calikoglu M, et al., Leptin and TNF-alpha levels in patients with chronic obstructive pulmonary disease and their relationship to nutritional parameters. Respiration. 2004 January-February; 71(1):45-50).

The expression of TNFR1 appears to be increased on peripheral T cells isolated from COPD patients (Hodge. G. et al., Increased intracellular T helper 1 proinflammatory cytokine production in peripheral blood, BAL and intraepithelial T cells of COPD subjects. Clin. Exp. Immunol. 2007. 150(1). 22-29).

Furthermore, elevated levels of peripheral soluble TNFR1 are strongly associated with morbidity and mortality in patients with acute lung injury and ventilator induced lung injury (Parsons, P. E. et al., Elevated plasma levels of soluble TNF receptors are associated with morbidity and mortality in patients with acute lung injury. Am J Physiol Lung Cell Mol. Physiol. 2005. 288: L426-L431).

Tumor necrosis factor alpha is a pleiotropic cytokine present at low concentrations in body fluids in pathological situations. It is a major mediator of immunological and pathophysiological reactions. TNF-α is produced mostly by activated macrophages and monocytes, but also by many other cell types including B lymphocytes, T lymphocytes and fibroblasts. TNF-α is normally synthesised as a 26 kDa precursor that is stored on cells as a membrane bound form. Prior to release from cells, the precursor form is converted to soluble 17 kDa TNF-α and activated by TNF-α converting enzyme (TACE) or other matrix metallo-proteinases.

TNF-α is a homotrimer which binds to two distinct cell surface receptors; the 55-60 kDa TNFR1 chain and the 70-80 kDa TNFR2 chain which both exist as non-covalently-bound homotrimeric receptor complexes. The TNFRs are expressed by a wide variety of cells. For example no cell type in the body has yet been found that does not express TNFR1 whereas expression of TNFR2 is mainly by immune cells and endothelial cells.

TNFR1 and TNFR2 are single transmembrane glycoproteins with 28% homology mostly in their extracellular domain and they contain four tandemly repeated cysteine rich motifs. They contain several motifs with known functional significance. Both TNFR1 and TNFR2 contain an extracellular pre-ligand-binding assembly domain (PLAD) domain (distinct from ligand binding regions) that pre-complexes receptors and promotes trimerization particularly upon activation by TNF-alpha ligand. TNFR1 contains a death domain (DD) motif of approximately 80 amino acids in length towards the carboxyl-end of the receptor and is critical in the death-inducing activity of TNFR1.

Binding studies at 0° C. defined high affinity binding of TNF-α to both TNFRs with K_{d} values of approximately 300-600 pM for TNFR1 and 70-200 pm to TNFR2 (MacEwan D J. et al., TNF receptor subtype signalling: differences and cellular consequences. Cell Signal. 2002 June; 14(6):477-92).

However at physiological temperatures TNFR1 (and not TNFR2) is the high affinity receptor for soluble TNF-α (K_{D}=19 pM) and this is driven mainly by a very slow off-rate of TNF-α for TNFR1. The complex of TNF-α with TNFR1 is very stable with a mean survival time of ˜48 minutes and is more likely to be internalised (than dissociate), leading to signalling complexes that persist intracellularly. Both TNFR1 and TNFR2 are also capable of binding lymphotoxin which is homologous to TNF-α (30% amino acid identity). The functional role of lymphotoxin is largely unknown in man.

Membrane-bound TNF-α binds to and activates both TNFR1 and TNFR2. Whilst, soluble TNF-α does bind TNFR2, subsequent signal transduction is significantly reduced compared to activation of TNFR2 by membrane-bound TNF-alpha. Once activated, TNFR2 is cleaved by matrix metalloproteinase (MMPs) into the soluble from which is still capable of binding to TNF-α ligand (Pennica D, et al., Biochemical properties of the 75-kDa tumor necrosis factor receptor. Characterization of ligand binding, internalization, and, receptor phosphorylation. J Biol. Chem. 1992 Oct. 15; 267(29):21172-8).

Multiple experimental approaches have revealed that TNF-R1 initiates the majority of biological activities of TNF-α. The binding of TNF-α to TNF-R1 triggers a series of intracellular events that ultimately results in the activation of two major transcription factors, nuclear factor kB (NF-kB) and c-Jun. These transcription factors are responsible for the inducible expression of genes important for diverse biological processes, including cell growth and death, development, oncogenesis, and immune, inflammatory, and stress responses. Transgenic mice deficient in TNFR1 display greater sensitivity to infection by Listeria monocytogenes or Mycobacterium tuberculosis but are resistant to TNF-α or interleukin-1-mediated in vivo lethality, plus were resistant to models of endotoxic shock induced by lipopolysaccharide and D-galactosamine. TNFR1 has also been shown to control early graph versus host disease.

Studies using transgenic mice deficient in TNFR2 have shown that TNFR2 plays an important role in several beneficial immunological processes. TNFR2 has been shown to play a role in antigen-driven T cell responses and proliferation. The type 1 receptor (CD120a) is the high-affinity receptor for soluble tumor necrosis factor. Proc Natl Acad Sci USA. 1998 Jan. 20; 95(2):570-5), anti tumor effects (Zhao X, et al., Tumor necrosis factor receptor 2-mediated tumor suppression is nitric oxide dependent and involves angiostasis. Cancer Res. 2007 May 1; 67(9):4443-50), ischemia-induced neovascularization (Goukassian D A, et al., Tumor necrosis factor-alpha receptor p75 is required in ischemia-induced neovascularization. Circulation. 2007 Feb. 13; 115(6):752-62), dendritic cell-natural killer cell crosstalk Langerhans cells migration. Finally, Higuchi Y et al., (Higuchi, Y. et al., Tumor Necrosis Factor Receptors 1 and 2 Differentially Regulate Survival, Cardiac Dysfunction, and Remodeling in Transgenic Mice With Tumor Necrosis Factor Induced Cardiomyopathy. Circulation. 2004; 109:1892-1897) showed that signalling via TNFR2 may play a cardioprotective role in the pathogenesis of cytokine-mediated heart failure. Specific blockade of TNFR1 could potentially allow TNF-α to exert some of its beneficial immune functions via signalling through TNFR2 whilst abrogating the detrimental effects of signalling through TNFR1.

Preclinical and clinical studies have identified and validated TNF-α as a key disease molecule and therapeutic target for immunotherapeutic intervention in many immune-mediated inflammatory diseases. Clinical indications include rheumatoid arthritis, Crohn's disease, ankylosing spondylitis and psoriasis. Recent clinical findings indicate that many chronic inflammatory disorders share certain pathogenic pathways, whereas others are limited to particular disease phenotypes. Using anti-TNFα agents as probes, it has been demonstrated that TNF-α regulates other pro-inflammatory cytokines such as IL-1α and IL-1β, inflammatory cell recruitment to joints, matrix metalloproteinases, and synovial vascularity (Taylor P C, et al., Tumour necrosis factor alpha as a therapeutic target for immune-mediated inflammatory diseases. Curr Opin Biotechnol. 2004 December; 15(6):557-63).

TNF-α may play a pivotal role in lung diseases such as COPD and asthma and amplifies the inflammatory response, resulting in activation of epithelial cells, monocytes, macrophages, and neutrophil mucus secretion and destruction of lung parenchyma through the release of proteinases.

COPD is characterised by slowly progressive development of airway limitation that is not fully reversible. COPD classically involves two spectra of clinical or pathological presentations, chronic bronchitis, and emphysema. While chronic bronchitis is defined clinically based on mucus production leading to cough with expectoration, emphysema is the pathological process of alveolar destruction. Both an increase in mucus cell hyperplasia and hypertrophy is apparent in the airways of patients diagnosed with COPD, in addition to a significant inflammatory infiltrate. While the dominant site of clinically relevant mucus secretion remains the larger airways, the majority of airflow obstruction is thought to reside in the non-cartilagenous, membranous bronchioles, and terminal airways. Prolonged inflammation in the small airways leads to sub-epithelial fibrosis, increased thickness of the bronchiole wall, and congestion of the lumen with mucus.

TNF-α is present at elevated concentrations in the induced sputum of COPD patients compared to normal smokers and asthma patients (Keatings V M, et al., Differences in interleukin-8 and tumor necrosis factor-alpha in induced sputum from patients with chronic obstructive pulmonary disease or asthma. Am J Respir Crit. Care Med. 1996 February; 153(2):530-4). In addition, increased expression of TNF-α may be temporally associated with exacerbation episodes in COPD patients (Calikoglu et al., 2004 as referenced earlier). While the expression of TNFR1 and TNFR2 in lung tissues does not appear to have been investigated, peripheral soluble TNFR2 levels have been shown to be increased in COPD patients, and expression of TNFR1 appears to be increased on peripheral T cells isolated from COPD patients (Hodge. G. et al., Increased intracellular T helper 1 proinflammatory cytokine production in peripheral blood, BAL and intraepithelial T cells of COPD subjects. Clin. Exp. Immunol. 2007. 150(1). 22-29). Furthermore, elevated levels of peripheral soluble TNFR1 are strongly associated with morbidity and mortality in patients with acute lung injury and ventilator induced lung injury (Parsons, P. E. et al., Elevated plasma levels of soluble TNF receptors are associated with morbidity and mortality in patients with acute lung injury. Am J Physiol Lung Cell Mol. Physiol. 2005. 288: L426-L431).

Serum concentrations of TNF-α and induced TNF-α production from peripheral monocytes are increased in COPD patients with weight loss and sarcopenia. This implicates TNF-α in the etiology of cachexia often associated with severe COPD.

Hence, anti-TNFR1 domain antibodies have been generated to inhibit the TNFR1 target (see for example the disclosure in WO2007049017 and WO06038027).

It is especially desirable to achieve local inhibition of the e.g. the TNFR1 target for treatment or prevention of pulmonary disease by direct pulmonary delivery of such anti-TNFR1 domain antibodies and hence a need exists for compositions which comprise agents which can inhibit the TNFR1 target e.g. anti-TNFR1 domain antibodies, which are especially suitable for direct administration to pulmonary tissues. Such compositions can be useful for treatment or prevention of a respiratory disease, e.g. COPD or asthma or pulmonary sarcoidosis.

The phrase “immunoglobulin single variable domain” refers to an antibody variable domain (V_{H}, V_{HH}, V_{L}) that specifically binds an antigen or epitope independently of other different V regions or domains. An immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of additional different variable domains). A “domain antibody” or “dAb” is the same as an “immunoglobulin single variable domain” as the term is used herein. In one embodiment, an immunoglobulin single variable domain is a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

### SUMMARY OF THE INVENTION

We have now developed compositions which comprise or consist of (a) a polypeptide, such as an antibody (e.g. a monoclonal antibody) or immunoglobulin polypeptide, for example a domain antibody (dAb), or e.g. a nanobody, and also (b) a pharmaceutically acceptable buffer, and wherein the composition comprises liquid droplets and about 40% or more e.g. 50% or more, of the liquid droplets present in the composition have a size in the range which is less than about 6 microns e.g. from about 1 micron to about 6 microns e.g. less than about 5 microns e.g. about 1 to about 5 microns These compositions are e.g. especially suitable for administration to a subject by direct local pulmonary delivery. These compositions can, for example, be administered directly to the lung, e.g. by inhalation, for example by using a nebuliser device.

Hence, the invention provides compositions comprising or consisting of (a) a polypeptide, such as an immunoglobulin polypeptide, e.g. a domain antibody (dAb) or e.g. a nanobody composition, and also (b) a pharmaceutically acceptable buffer, and wherein the composition comprises liquid droplets and about 40% or more, e.g. 50% or more, of the liquid droplets present in the composition have a size in the range which is less than about 6 microns, e.g. from about 1 micron to about 6 microns e.g. less than about 5 microns or e.g. about 1 to about 5 microns e.g. for administration to a subject by direct local pulmonary delivery. These compositions can comprise a physiologically acceptable buffer, which has a pH range of between about 4 to about 8, e.g. about 7 to about 7.5, and a viscosity which is about equal to the viscosity of a solution of about 2% to about 10% PEG 1000 in 50 mM phosphate buffer containing 1.2% (w/v) sucrose.

At least 40%, e.g. 50% or more, e.g. 80% or more of the liquid droplets present in the composition have a size in the range which is less than about 6 microns e.g. from about 1 micron to about 6 microns e.g. 80% or more. The size range can be as mentioned less than about 6 microns e.g. from about 1 micron to about 6 microns, e.g. from about 2 to about 5 microns, and for delivery to the deep lung it can preferably be for example from about 2 to about 3 microns.

Examples of currently known pharmaceutically acceptable buffers include phosphate, citrate, acetate and histidine buffers. The buffer can also comprise additional agents e.g. (a) agents to increase the viscosity such as PEG, e.g. PEG 1000; sugars e.g. sucrose, mannose, lutrol e.g. lutrol 44 and/or (b) stabilising agents such as detergents.

The invention further relates to compositions as described above which also further comprise a pharmaceutically acceptable carrier, diluent or excipient.

The polypeptides can comprise or consist of e.g. therapeutic, prophylactic or diagnostic polypeptides, which it is desirable to deliver to a subject, e.g. of up to about 150 amino acids.

The polypeptides can be for example antibody (e.g. a monoclonal antibody) or immunoglobulin polypeptides, or they can be e.g. polypeptide domains such as monomers, e.g. of up to about 150 amino acids.

The polypeptides can comprise or consist of for example domain antibodies (“dAbs”) e.g. domain antibody (dAb) monomers.

The polypeptides can also comprise or consist of non-IgG like scaffolds, such as an affibody.

The term “polypeptide” or domain antibody (“dAb”) as used herein is also used to refer to for example polypeptides or dAbs which are fused to or conjugated to or associated with other molecules, e.g. which it is desirable to deliver to a subject e.g. to Albudabs, wherein the dAbs are associated with human serum albumin to extend the half life (see for example WO2005118642 and WO 2006059106 the teachings of which are incorporated herein by reference)

The polypeptides, e.g. domain antibody molecules, such as dAb monomers, can bind to a desired target, e.g. a target present in pulmonary tissue, and for example the target can be one which plays a role in a lung condition or lung disease or lung disorder, e.g. the target can be the TNF receptor e.g. TNFR1. The polypeptides can also bind to more than one target, e.g. they can be dual targeting dAbs such as those described in WO2004058821.

The dAbs can bind any target molecule of interest. The dAbs can also be formatted dAbs e.g. they can be dAb-FC fusions or they can be attached to another group e.g. a PEG group. The dAbs can also be ones which are linked to another molecule e.g. as a fusion e.g. to another molecule which binds to a target.

The invention also relates to use of the compositions as described above e.g. a domain antibody (dAb) composition, e.g. that binds a target in pulmonary tissue, for administration to a subject by direct local pulmonary delivery.

Also provided is a composition as described herein, e.g. a dAb composition as described herein, for use in medicine, e.g. for the treatment, prevention or diagnosis of a respiratory condition or lung disease or disorder, e.g. COPD or asthma or pulmonary sarcoidosis.

The invention also relates to use of a composition, e.g. a dAb composition as described above, in the manufacture of medicament for treatment, prevention or diagnosis of a respiratory condition or lung disease or disorder. In one embodiment, up to about 10 mg of a dAb that binds a target in pulmonary tissue can be used. The target in pulmonary tissue can be one which mediates lung inflammation or a pulmonary disease.

Also provided is a method for treating, preventing or diagnosing a disease or condition e.g. a respiratory condition or lung disease or disorder, which comprises administering to a subject an amount e.g. a therapeutically effective amount of a composition described herein, such as a dAb composition. The dosage administered can range from about 5 mg per Kg to about 0.005 mg per Kg, e.g. about 0.5 mg/Kg to about 0.1 mg/Kg body weight of a subject as a daily dosage. In certain embodiments the composition is administered directly to the lung tissue, e.g. by inhalation, e.g. by using a nebuliser, intranasal or inhalation device.

The invention further relates to a delivery device, for example a nebuliser, inhaler or intranasal delivery device, and its use for providing a dose e.g. a metered dose, of the compositions described herein, e.g. a dAb composition, to a subject for the treatment or prevention or diagnosis of a disease or condition, e.g. a respiratory disease or condition, wherein the inhaler or intranasal delivery device comprises the dAb formulation and for example provides a metered daily dose, for example containing up to 10 mg of dAb. Different types of nebuliser devices can be used according to the invention, e.g. jet nebuliser devices such as PARI, and e.g. vibrating mesh nebulisers such as eFlow and Aeroneb, and also e.g. ultrasonic devices such as DeVilbiss and Kun-88

Also provided by the invention is a process for producing the compositions of the invention which comprises the steps of mixing (a) a polypeptide e.g. a domain antibody (dAb) with (b) a pharmaceutically acceptable buffer, e.g. a buffer which has a pH range of between about 4 to about 8, e.g. about 7 to about 7.5, and a viscosity which is about equal to the viscosity of a solution of about 2% to about 10% PEG 1000 in 50 mM phosphate buffer containing 1.2% (w/v) sucrose. Additional agents can also be added, e.g. a pharmaceutically acceptable diluent carrier or excipient, and/or agents to increase viscosity, and/or stabilising agents.

The invention also relates to use of compositions described herein e.g. domain antibody (dAb) compositions described herein for diagnostic purposes (e.g. for imaging), wherein the heavy or light chain portion from an immunoglobulin like molecule e.g. the domain antibody, can advantageously comprise a detectable label. Suitable detectable labels and methods for labelling an agent are well known in the art. Suitable detectable labels include, for example, a radioisotope (e.g., as Indium-111, Technetium-99m or Iodine-131), positron emitting labels (e.g., Fluorine-19), paramagnetic ions (e.g., Gadlinium (III), Manganese (II)), an epitope label (tag), an affinity label (e.g., biotin, avidin), a spin label, an enzyme, a fluorescent group or a chemiluminescent group. When labels are not employed, complex formation can be determined by surface plasmon resonance or other suitable methods.

The invention also relates to use of compositions described herein, e.g. domain antibody (dAb) compositions as described herein, in the manufacture of a nebuliser, inhaler or intranasal delivery device, for the purpose of providing a long-acting inhaled dAb formulation for local delivery to the lung.

The invention also relates to a method for administering the compositions e.g. domain antibody (dAb) compositions described herein and that bind a target in pulmonary tissue, to a subject to produce a long therapeutic window in pulmonary tissue, comprising administering locally to pulmonary tissue of said subject an effective amount of said compositions e.g. said domain antibody (dAb) compositions as described herein.

The invention also relates to a process for producing a polypeptide composition such as a dAb composition, e.g. for treating, preventing or diagnosing a lung condition or disease which comprises mixing (a) a polypeptide with (b) a physiologically acceptable buffer which has a pH range of between about 4 and about 8 and a viscosity which is about equal to the viscosity of a solution of about 2% to about 10% PEG 1000 in 50 mM phosphate buffer containing 1.2% (w/v) sucrose.

The invention further relates to a process for producing a polypeptide composition such as a dAb composition which comprises the steps of (a) mixing a polypeptide with a physiologically acceptable buffer, such as a buffer which has a pH range of between about 4 and about 8 and a viscosity which is about equal to the viscosity of a solution of about 2% to about 10% PEG 1000 in 50 mM phosphate buffer containing 1.2% (w/v) sucrose, and then (b) passing the polypeptide and buffer composition from step (a) through a nebuliser, inhaler or intranasal delivery device.

The invention still further relates to use of a physiologically acceptable buffer which has a pH range of between about 4 and about 8 and a viscosity which is about equal to the viscosity of a solution of about 2% to about 10% PEG 1000 in 50 mM phosphate buffer containing 1.2% (w/v) sucrose, for the manufacture of a polypeptide composition such as a dAb composition, e.g. for pulmonary delivery.

The invention also relates to methods for delivering desired molecules into the systemic circulation, which comprises first administering the compositions described herein to the lung by inhalation e.g. using a nebuliser, intranasal or inhalation device.

The invention also relates to dAbs described herein and to compositions as described herein comprising these dAbs, to uses of the dAbs as described above, and to methods for making these dAb compositions, and also to devices e.g. nebuliser or inhaler devices as described above, which comprise these dAb compositions.

### BRIEF DESCRIPTION OF THE DRAWINGS

FIG. 1: shows the amino acid sequences of the Dom1h-131 lead domain antibodies against TNFR1.

FIG. 2: shows the TNF-alpha dose curve in the human TNFR1 receptor binding assay. Each sample was tested as four replicates.

FIG. 3: shows Inhibition by DOM1H-131-202, DOM1H-131-206 and DOM1H-131-511 in the human TNFR1 receptor binding assay. Each sample was tested as four replicates.

FIG. 4: shows Protease stability data for DOM1H-131-202, DOM1H-131-511 and DOM1H-131-206

FIG. 5: shows Effect of buffer and device on nebulised droplet size of GSK 1995056A (511)

FIG. 6: shows Stability of DOM1H-131-511 (511) after nebulisation in various devices assessed by dimer formation measured by SEC.

FIG. 7: shows SEC traces of 206 AT 40 mg/ml for up to 1 hour using the Pari LC device

FIG. 8: shows RBA results showing DOM1H-131-511 (511) retains activity after nebulisation.

FIG. 9: shows Nebuliser testing of DOM1H-131-202 (202), DOM1H-131-206 (206) and DOM1H-131-511 (511) in the Pari E-flow and LC+.

The protein concentration was 5 mg/ml in either Britton-Robinson or formulation buffer. The standard (drug X) was tested in its own formulation buffer for delivery as a control.

FIG. 10: Shows SEC traces for DOM10-53-474 pre- and post nebulisation using a vibrating mesh nebuliser (E-flow, Pari).

FIG. 11: shows SEC traces for DOM10-53-474 pre- and post nebulisation using a jet nebuliser (LC+, Pari).

FIG. 12 a-e: shows the Amino acid sequences of (a) Exendin 4 (G4S)3 DOM7h-14 fusion (DAT0115), (b) DOM10-53-474 (anti IL-13 dAb) (c) DOM10-275-78 (anti IL-13 dAb) (d) DOM4-130-202 (anti-IL-1R1) and (e) DOM4-130-201 (anti-IL-1R1).

FIG. 13 a-d: shows the Amino acid sequences of certain anti TNFR1 dAbs (a) Dom 1 h-131-201 (b) Dom 1h-131-203 (c) Dom 1h-131-204 (d) Dom 1h-131-205.

FIG. 14 a-i: shows the Amino acid sequences of certain anti VEGF dAbs (a) Dom 15-26-593(b) Dom 15-26-501 (c) Dom 15-26-555 (d) Dom 15-26-558 (e) Dom 15-26-589 (f) Dom 15-26-591 dAbs (g) Dom 15-26-594 (h) Dom 15-26-595 (i) DMS1529 (VEGF dAb 15-26-593-Fc Fusion).

### DETAILED DESCRIPTION OF THE INVENTION

### Definitions

The phrase “immunoglobulin single variable domain” refers to an antibody variable domain (V_{H}, V_{HH}, V_{L}) that specifically binds an antigen or epitope independently of other V regions or domains. An immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” as the term is used herein. In certain embodiments an immunoglobulin single variable domain is a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The term “polypeptide” refers to any kind of polypeptide such as peptides, human proteins, fragments of human proteins, proteins or fragments of proteins from non-human sources, engineered versions proteins or fragments of proteins, enzymes, antigens, drugs, molecules involved in cell signalling, such as receptor molecules, antibodies, including polypeptides of the immunoglobulin superfamily, such as antibody polypeptides or T-cell receptor polypeptides.

The polypeptides can be for example antibody or immunoglobulin polypeptides, or they can be e.g. polypeptide domains such as monomers, e.g. of up to about 150 amino acids.

The polypeptides can usefully comprise or consist of for example domain antibodies (“dAbs”) e.g. dAb monomers.

The polypeptides can also comprise or consist of non-IgG like scaffolds, such as an affibody.

The term “polypeptide” or domain antibody (“dAb”) as used herein is also used to refer to for example polypeptides or dAbs which are fused to or conjugated or associated with other molecules. For example the polypeptides e.g. dAbs may be Pegylated and Pegylated dAbs are for example described in WO2004081026. The polypeptides e.g. dAbs, may be associated with serum albumin e.g. they can be the dAbs linked to serum albumin (Albudabs) described in WO2005118642 and WO2006059106.

Advantageously, the antibody polypeptides may comprise both heavy chain (V_{H}) and light chain (V_{L}) polypeptides, or single domain antibody repertoires comprising either heavy chain (V_{H}) or light chain (V_{L}) polypeptides. An antibody polypeptide, as used herein, is a polypeptide which either is an antibody or is a part of an antibody, modified or unmodified. Thus, the term antibody polypeptide includes a heavy chain, a light chain, a heavy chain-light chain dimer, a Fab fragment, a F(ab′)₂ fragment, a heavy chain single domain, a light chain single domain, a Dab fragment, or an Fv fragment, including a single chain Fv (scFv). Methods for the construction of such antibody molecules and nucleic acids encoding them are well known in the art.

Compositions as described herein comprising polypeptides such as domain antibodies, can bind a target in pulmonary tissue selected from the group consisting of TNFR1, IL-1, IL-1R, IL-4, IL-4R, IL-5, IL-6, IL-6R, IL-8, IL-8R, IL-9, IL-9R, IL-10, IL-12 IL-12R, IL-13, IL-13Rα1, IL-13Rα2, IL-15, IL-15R, IL-16, IL-17R, IL-17, IL-18, IL-18R, IL-23 IL-23R, IL-25, CD2, CD4, CD11a, CD23, CD25, CD27, CD28, CD30, CD40, CD40 L, CD56, CD138, ALK5, EGFR, FcER1, TGFb, CCL2, CCL18, CEA, CRS, CTGF, CXCL12 (SDF-1), chymase, FGF, Furin, Endothelin-1, Eotaxins (e.g., Eotaxin, Eotaxin-2, Eotaxin-3), GM-CSF, ICAM-1, ICOS, IgE, IFNa, 1-309, integrins, L-selectin, MIF, MIP4, MDC, MCP-1, MMPs, neutrophil elastase, osteopontin, OX-40, PARC, PD-1, RANTES, SCF, SDF-1, siglec8, TARC, TGFb, Thrombin, Tim-1, TNF, TNFR1, TRANCE, Tryptase, VEGF, VLA-4, VCAM, α4β7, CCR2, CCR3, CCR4, CCR5, CCR7, CCR8, alphavbeta6, alphavbeta 8, cMET, and CD8.

Compositions as described herein comprising polypeptides such as domain antibodies, can also bind systemic targets for example targets can be GLP-1, Exendin and Interferon.

In an embodiment, compositions as described herein comprising polypeptides such as domain antibodies can bind a target selected from the group consisting of a protein in the TNF signalling cascade. In certain embodiments, this protein target is selected from the group comprising TNF alpha, TNF beta, TNFR2, TRADD, FADD, Caspase-8, TNF receptor-associated factor (TRAF), TRAF2, receptor-interacting protein (RIP), Hsp90, Cdc37, IKK alpha, IKK beta, NEMO, inhibitor of kB (IkB), NF-kB, NF-kB essential modulator, apoptosis signal-regulated kinase-1 (aSMase), neutral sphingomyelinase (nSMase), ASK1, Cathepsin-B, germinal center kinase (GSK), GSK-3, factor-associated death domain protein (FADD), factor associated with neutral sphingomyelinase activation (FAN), FLIP, JunD, inhibitor of NF-kB kinase (IKK), MKK3, MKK4, MKK7, IKK gamma, mitogen-activated protein kinase/Erk kinase kinase (MEKK), MEKK1, MEKK3, NIK, poly(ADP-ribose) polymerase (PARP), PKC-zeta, RelA, T2K, TRAF1, TRAF5, death effector domain (DED), death domain (DD), death inducing signalling complex (DISC), inhibitor of apoptosis protein (IAP), c-Jun N-erminal kinase (JNK), mitogen-activated protein kinase (MAPK), phosphoinositide-3OH kinase (PI3K), protein kinase A (PKA), PKB, PKC, PLAD, PTEN, rel homology domain (RHD), really interesting new gene (RING), stress-activated protein kinase (SAPK), TNF alpha-converting enzyme (TACE), silencer of death domain protein (SODD), and TRAF-associated NF-kB activator (TANK). With regard to these preferred targets, reference is made to WO04046189, WO04046186 and WO04046185 (incorporated herein by reference) which provide guidance on the selection of antibody single variable domains for targeting intracellular targets.

The invention particularly relates to an antagonist of TNFR1 which is a domain antibody formulated as described herein and to its use in the manufacture of a medicament for treating, suppressing or preventing lung inflammation and/or a respiratory disease e.g. COPD or asthma.

Also provided are compositions as described herein for pulmonary delivery which comprise a molecule e.g. a dAb, which binds to IL-13, e.g. for use in treating asthma. Examples of these dAbs are described in for example WO2007/085815 and also herein as DOM10-275-78 and DOM10-275-78.

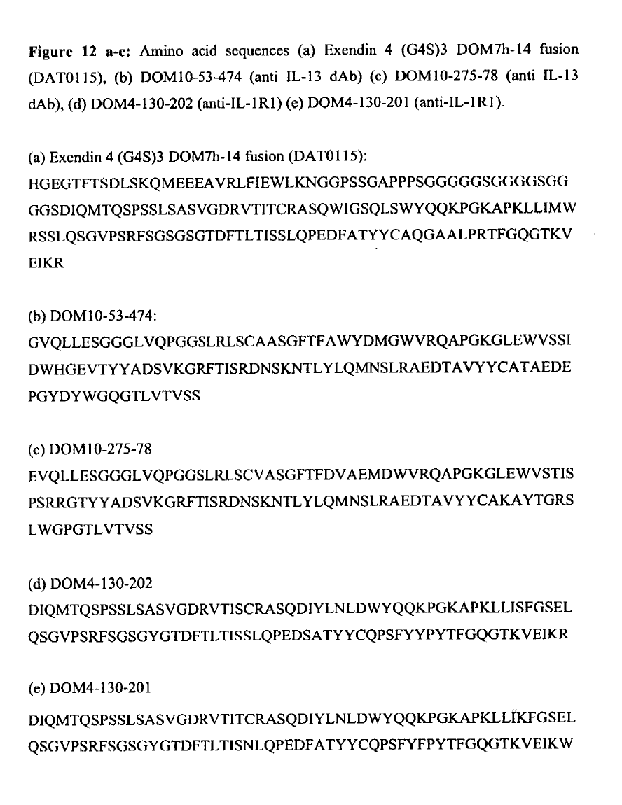

The invention further provides compositions as described herein for pulmonary delivery comprising an immunoglobulin single variable domain that binds IL-13 e.g. for treating asthma, and that has an amino acid sequence which is identical to an amino acid sequence disclosed in FIG. 12b (Dom 10-53-474) or FIG. 12c (Dom 10-275-78) or which has e.g. 80% identity with an amino acid sequence disclosed in FIG. 12b or FIG. 12c, e.g. 85%, 86%, 87%, 88%, 89%, 90%, 91%, 92%, 93%, 94% 95%, 96% or 97% identity.

The invention further provides compositions as described herein for pulmonary delivery comprising an immunoglobulin single variable domain that binds IL-1R1 e.g. for treating an inflammatory condition e.g. lung inflammation or lung disease or Rheumatoid arthritis, and that has an amino acid sequence which is identical to an amino acid sequence disclosed in FIG. 12d (Dom 4-130-202) or FIG. 12e (Dom 4-130-201) or which has e.g. 80% identity with an amino acid sequence disclosed in FIG. 12d or FIG. 12e, e.g. 85%, 86%, 87%, 88%, 89%, 90%, 91%, 92%, 93%, 94% 95%, 96% or 97% identity.

Also provided are compositions as described herein for pulmonary delivery comprising an amino acid sequence that is at least 97% identical to the amino acid sequence of Dom 4-130-202 (shown in FIG. 12d).

Also provided are compositions as described herein for pulmonary delivery comprising an amino acid sequence that is at least 98% identical to the amino acid sequence of Dom 4-130-202 (shown in FIG. 12e).

Also provided are compositions as described herein for pulmonary delivery which comprise a molecule e.g. a dAb, which binds to VEGF e.g. for treating cancer, for example any of those described in WO2007080392 and WO2007066106.

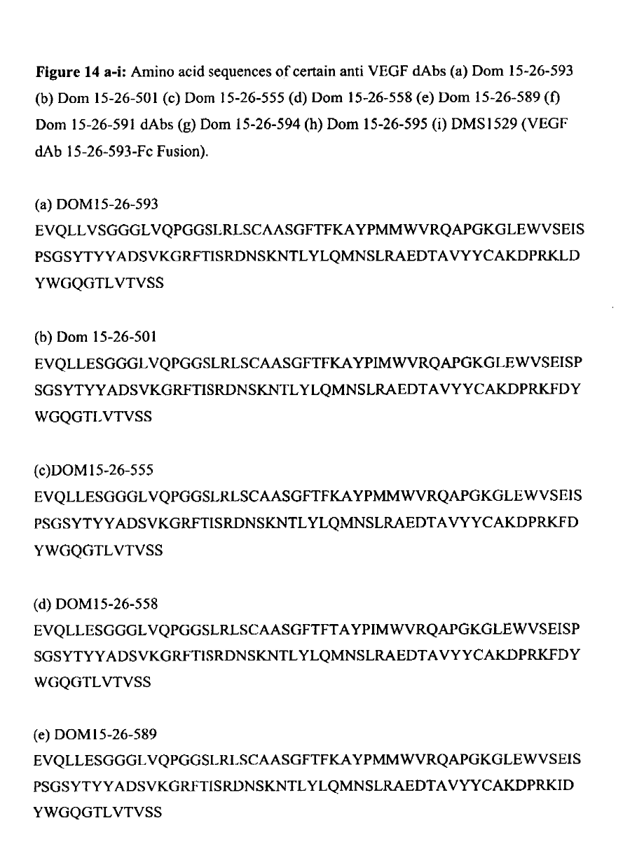

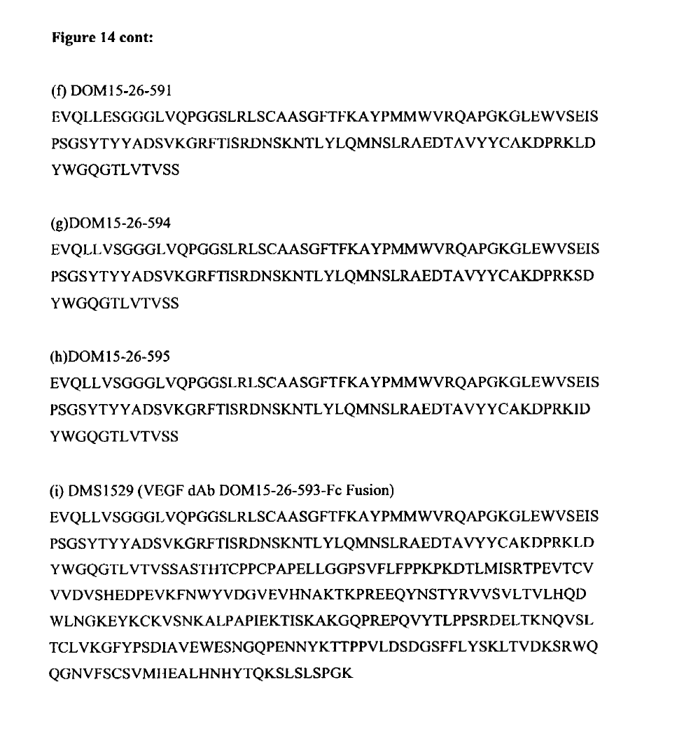

The invention further provides compositions as described herein for pulmonary delivery comprising an immunoglobulin single variable domain that binds VEGF e.g. for treating cancer, and that has an amino acid sequence which is identical to an amino acid sequence disclosed in FIG. 14 or which has e.g. 80% identity with an amino acid sequence disclosed in FIG. 14, e.g. 85%, 86%, 87%, 88%, 89%, 90%, 91%, 92%, 93%, 94% 95%, 96% or 97% or 98%, 99% identity.

Also provided are compositions as described herein for pulmonary delivery which comprise an anti-VEGF immunoglobulin single variable domain comprising an amino acid sequence that is at least 97% identical to the amino acid sequence of DOM15-26-593 (shown in FIG. 14a).

Also provided are compositions as described herein for pulmonary delivery which comprise an anti-VEGF immunoglobulin single variable domain comprising an amino acid sequence that is at least 97% identical to the amino acid sequence of DOM15-26-593 (shown in FIG. 14a), and which further comprises a domain of an antibody constant region.

The inventions also provides compositions as described herein for pulmonary delivery which comprise an anti-VEGF immunoglobulin single variable domain comprising an amino acid sequence that is selected from the amino acid sequence of DOM15-26-593 (shown in FIG. 14a) and the amino acid sequence of DOM15-26-593-Fc Fusion (DMS1529; shown in FIG. 14i)

The compositions of the invention are especially suitable for direct delivery to the lung and hence the compositions can be used to treat, suppress, prevent or diagnose lung or respiratory conditions or diseases e.g. at or near to the site of delivery.

However, the compositions can first be delivered to the lung but used to treat diseases in other parts of the body e.g. systemic diseases. This is because the compositions initially delivered to the lung may then be absorbed into the systemic circulation, hence enabling treatment of diseases which are other than lung diseases. For example molecules which bind to the GLP receptor can be delivered to the lung and these can be used to treat diseases such as diabetes or obesity. Examples of these molecules include those described in WO2006/059106 and the exendin-4 linked to an albudab described herein as Exendin 4 (G4S)3 DOM7h-14 fusion (DAT0115) or any molecule which has e.g. 80% identity with dat0115 amino acid sequence e.g. 85%, 90%, 91%, 92%, 93%, 94% 95%, 96% or 97%, 98%, or 99% identity.

Respiratory conditions or diseases that can be treated, suppressed or prevented using the medicaments, compositions and formulations and methods of the invention include lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, Wegener's granulomatosis, and non-small cell lung carcinoma.

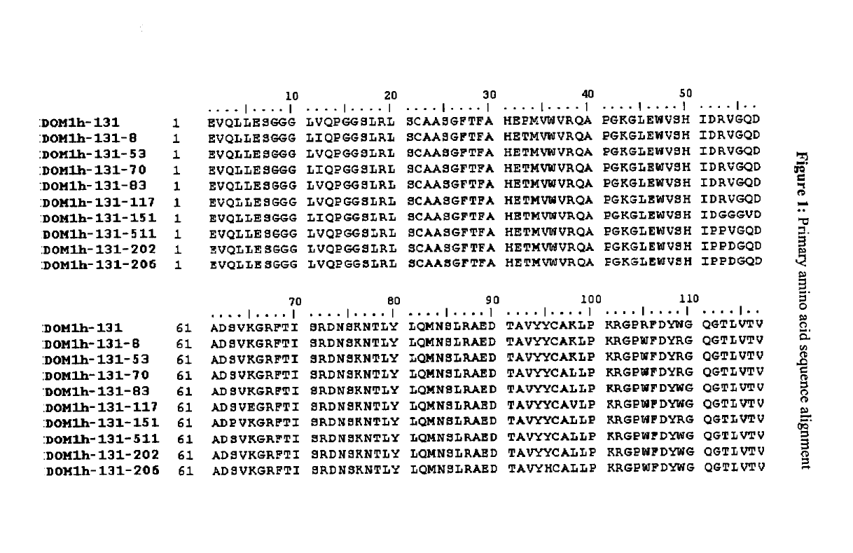

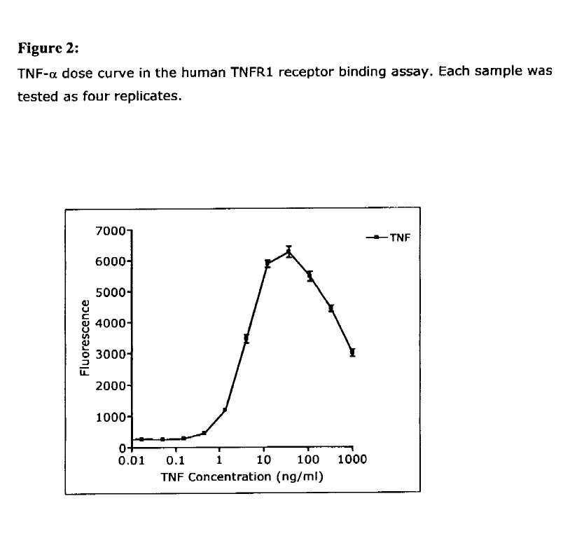

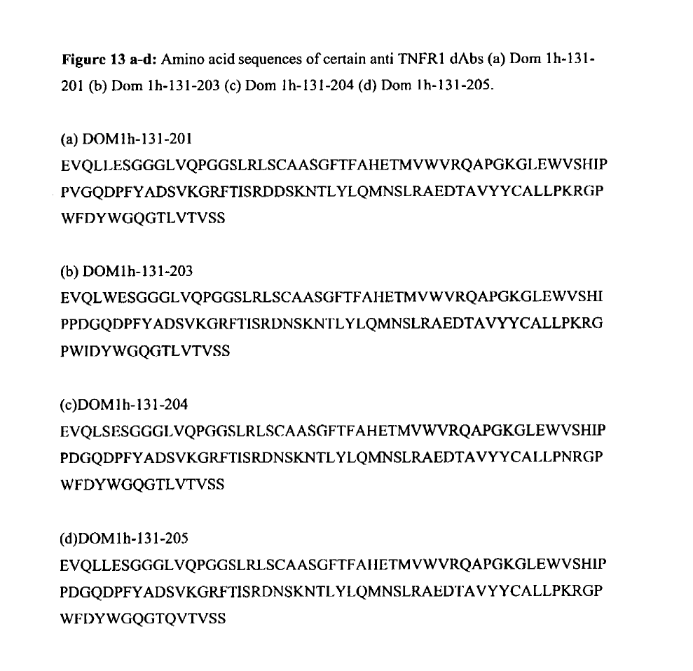

Hence the invention provides compositions as described herein for pulmonary delivery comprising an immunoglobulin single variable domain that binds TNFR1 and that has an amino acid sequence which is identical to an amino acid sequence disclosed in FIG. 1 or FIG. 13 or which has e.g. 80% identity with an amino acid sequence disclosed in FIG. 1 or FIG. 13, e.g. 85%, 86%, 87%, 88%, 89%, 90%, 91%, 92%, 93%, 94% 95%, 96% or 97% identity.

The invention also provides compositions as described herein for pulmonary delivery comprising an anti-TNFα receptor type 1 (TNFR1; p55) immunoglobulin single variable domain comprising an amino acid sequence that is at least 93% identical to the amino acid sequence of DOM1h-131-206 (shown in FIG. 1).

The invention also provides compositions as described herein for pulmonary delivery comprising an anti-TNFα receptor type 1 (TNFR1; p55) immunoglobulin single variable domain comprising an amino acid sequence that is at least 95% identical to the amino acid sequence of DOM1h-131-511 (shown in FIG. 1).

Domain antibodies which can be used in the compositions of the invention can comprise a immunoglobulin single variable domain that binds TNFR1, wherein the immunoglobulin single variable domain that binds TNFR1 differs from the amino acid sequence of an anti-TNFR1 dAb disclosed herein at no more than 25 amino acid positions and has a CDR1 sequence that has at least 50% identity to the CDR1 sequence of the anti-TNFR1 dAbs disclosed herein. Domain antibodies which can be used in the compositions of the invention can comprise an immunoglobulin single variable domain that binds TNFR1, wherein the amino acid sequence of the immunoglobulin single variable domain that binds TNFR1 differs from the amino acid sequence of an anti-TNFR1 dAb disclosed herein at no more than 25 amino acid positions and has a CDR2 sequence that has at least 50% identity to the CDR2 sequence of the anti-TNFR1 dAbs disclosed herein.

Domain antibodies which can be used in the compositions of the invention can comprise can comprise an immunoglobulin single variable domain that binds TNFR1, wherein the amino acid sequence of the immunoglobulin single variable domain that binds TNFR1 differs from the amino acid sequence of an anti-TNFR1 dAb disclosed herein at no more than 25 amino acid positions and has a CDR3 sequence that has at least 50% identity to the CDR3 sequence of the anti-TNFR1 dAbs disclosed herein.

Domain antibodies which can be used in the compositions of the invention can comprise can comprise an immunoglobulin single variable domain that binds TNFR1, wherein the amino acid sequence of the immunoglobulin single variable domain that binds TNFR1 differs from the amino acid sequence of an anti-TNFR1 dAb disclosed herein at no more than 25 amino acid positions and has a CDR1 sequence and a CDR2 sequence that has at least 50% identity to the CDR1 or CDR2 sequences, respectively, of the anti-TNFR1 Abs disclosed herein.

Domain antibodies which can be used in the compositions of the invention can comprise can comprise an immunoglobulin single variable domain that binds TNFR1, wherein the amino acid sequence of the immunoglobulin single variable domain that binds TNFR1 differs from the amino acid sequence of an anti-TNFR1 dAb disclosed herein at no more than 25 amino acid positions and has a CDR2 sequence and a CDR3 sequence that has at least 50% identity to the CDR2 or CDR3 sequences, respectively, of the anti-TNFR1 dAbs disclosed herein.

Domain antibodies which can be used in the compositions of the invention can comprise can comprise an immunoglobulin single variable domain that binds TNFR1, wherein the amino acid sequence of the immunoglobulin single variable domain that binds TNFR1 differs from the amino acid sequence of an anti-TNFR1 dAb disclosed herein at no more than 25 amino acid positions and has a CDR1 sequence and a CDR3 sequence that has at least 50% identity to the CDR1 or CDR3 sequences, respectively, of the anti-TNFR1 dAbs disclosed herein.

Domain antibodies which can be used in the compositions of the invention can comprise can comprise an immunoglobulin single variable domain that binds TNFR1, wherein the amino acid sequence of the immunoglobulin single variable domain that binds TNFR1 differs from the amino acid sequence of an anti-TNFR1 dAb disclosed herein at no more than 25 amino acid positions and has a CDR1 sequence, a CDR2 sequence and a CDR3 sequence that has at least 50% identity to the CDR1, CDR2 or CDR3 sequences, respectively, of the anti-TNFR1 dAbs disclosed herein.

Domain antibodies which can be used in the compositions of the invention can comprise can comprise an immunoglobulin single variable domain that binds TNFR1, wherein the immunoglobulin single variable domain that binds TNFR1 has a CDR1 sequence that has at least 50% identity to the CDR1 sequences of an anti-TNFR1 dAb disclosed herein.

Domain antibodies which can be used in the compositions of the invention can comprise can comprise an immunoglobulin single variable domain that binds TNFR1, wherein the immunoglobulin single variable domain that binds TNFR1 has a CDR2 sequence that has at least 50% identity to the CDR2 sequences of an anti-TNFR1 dAb disclosed herein.

Domain antibodies which can be used in the compositions of the invention can comprise can comprise an immunoglobulin single variable domain that binds TNFR1, wherein the immunoglobulin single variable domain that binds TNFR1 has a CDR3 sequence that has at least 50% identity to the CDR3 sequences of an anti-TNFR1 dAb disclosed herein.

Domain antibodies which can be used in the compositions of the invention can comprise can comprise an immunoglobulin single variable domain that binds TNFR1, wherein the immunoglobulin single variable domain that binds TNFR1 has a CDR1 and a CDR2 sequence that has at least 50% identity to the CDR1 and CDR2 sequences, respectively, of an anti-TNFR1 dAb disclosed herein. Domain antibodies which can be used in the compositions of the invention can comprise can comprise an immunoglobulin single variable domain that binds TNFR1, wherein the immunoglobulin single variable domain that binds TNFR1 has a CDR2 and a CDR3 sequence that has at least 50% identity to the CDR2 and CDR3 sequences, respectively, of an anti-TNFR1 dAb disclosed herein.

Domain antibodies which can be used in the compositions of the invention can comprise can comprise an immunoglobulin single variable domain that binds TNFR1, wherein the immunoglobulin single variable domain that binds TNFR1 has a CDR1 and a CDR3 sequence that has at least 50% identity to the CDR1 and CDR3 sequences, respectively, of an anti-TNFR1 dAb disclosed herein.

Domain antibodies which can be used in the compositions of the invention can comprise can comprise an immunoglobulin single variable domain that binds TNFR1, wherein the immunoglobulin single variable domain that binds TNFR1 has a CDR1, CDR2, and a CDR3 sequence that has at least 50% identity to the CDR1, CDR2 and CDR3 sequences, respectively, of an anti-TNFR1 dAb disclosed herein.

Polypeptides, immunoglobulin single variable domains and antagonists which are used in compositions of the invention for pulmonary delivery may be resistant to one or more of the following: serine protease, cysteine protease, aspartate proteases, thiol proteases, matrix metalloprotease, carboxypeptidase (e.g., carboxypeptidase A, carboxypeptidase B), trypsin, chymotrypsin, pepsin, papain, elastase, leukozyme, pancreatin, thrombin, plasmin, cathepsins (e.g., cathepsin G), proteinase (e.g., proteinase 1, proteinase 2, proteinase 3), thermolysin, chymosin, enteropeptidase, caspase (e.g., caspase 1, caspase 2, caspase 4, caspase 5, caspase 9, caspase 12, caspase 13), calpain, ficain, clostripain, actimidain, bromelain, and separase. In particular embodiments, the protease is trypsin, elastase or leucozyme. The protease can also be provided by a biological extract, biological homogenate or biological preparation. In one embodiment, the protease is a protease found in sputum, mucus (e.g., gastric mucus, nasal mucus, bronchial mucus), bronchoalveolar lavage, lung homogenate, lung extract, pancreatic extract, gastric fluid, saliva. In one embodiment, the protease is one found in the eye and/or tears. Examples of such proteases found in the eye include caspases, calpains, matric metalloproteases, disintegrin, metalloproteinases (ADAMs) and ADAM with thrombospondin mitifs, the proteosomes, tissue plasminogen activator, secretases, cathepsin B and D, cystatin C, serine protease PRSS1, ubiquitin proteosome pathway (UPP). In one embodiment, the protease is a non-bacterial protease. In an embodiment, the protease is an animal, eg, mammalian, eg, human, protease. In an embodiment, the protease is a GI tract protease or a pulmonary tissue protease, eg, a GI tract protease or a pulmonary tissue protease found in humans. Such protease listed here can also be used in the methods described herein involving exposure of a repertoire of library to a protease.

In one aspect, the composition of the invention comprise a protease resistant immunoglobulin single variable domain, wherein the variable domain is resistant to protease when incubated with i) a concentration (c) of at least 10 micrograms/ml protease at 37° C. for time (t) of at least one hour;or (ii) a concentration (c′) of at least 40 micrograms/ml protease at 30° C. for time (t) of at least one hour. In one embodiment, the ratio (on a mole/mole basis) of protease, eg trypsin, to variable domain is 8,000 to 80,000 protease:variable domain, eg when C is 10 micrograms/ml, the ratio is 800 to 80,000 protease:variable domain; or when C or C′ is 100 micrograms/ml, the ratio is 8,000 to 80,000 protease:variable domain. In one embodiment the ratio (on a weight/weight, eg microgram/microgram basis) of protease (eg, trypsin) to variable domain is 16,000 to 160,000 protease:variable domain eg when C is 10 micrograms/ml, the ratio is 1,600 to 160,000 protease:variable domain; or when C or C′ is 100 micrograms/ml, the ratio is 1,6000 to 160,000 protease:variable domain. In one embodiment, the concentration (c or c′) is at least 100 or 1000 micrograms/ml protease. In one embodiment, the concentration (c or c′) is at least 100 or 1000 micrograms/ml protease. Reference is made to the description herein of the conditions suitable for proteolytic activity of the protease for use when working with repertoires or libraries of peptides or polypeptides (eg, w/w parameters). These conditions can be used for conditions to determine the protease resistance of a particular immunoglobulin single variable domain. In one embodiment, time (t) is or is about one, three or 24 hours or overnight (e.g., about 12-16 hours). In one embodiment, the variable domain is resistant under conditions (i) and the concentration (c) is or is about 10 or 100 micrograms/ml protease and time (t) is 1 hour. In one embodiment, the variable domain is resistant under conditions (ii) and the concentration (c′) is or is about 40 micrograms/ml protease and time (t) is or is about 3 hours. In one embodiment, the protease is selected from trypsin, elastase, leucozyme and pancreatin. In one embodiment, the protease is trypsin. In one embodiment, the protease is a protease found in sputum, mucus (e.g., gastric mucus, nasal mucus, bronchial mucus), bronchoalveolar lavage, lung homogenate, lung extract, pancreatic extract, gastric fluid, saliva or tears or the eye. In one embodiment, the protease is one found in the eye and/or tears. In one embodiment, the protease is a non-bacterial protease. In an embodiment, the protease is an animal, eg, mammalian, eg, human, protease. In an embodiment, the protease is a GI tract protease or a pulmonary tissue protease, eg, a GI tract protease or a pulmonary tissue protease found in humans. Such protease listed here can also be used in the methods described herein involving exposure of a repertoire of library to a protease.

In one embodiment, the variable domain is resistant to trypsin and/or at least one other protease selected from elastase, leucozyme and pancreatin. For example, resistance is to trypsin and elastase; trypsin and leucozyme; trypsin and pacreatin; trypsin, elastase and leucozyme; trypsin, elastase and pancreatin; trypsin, elastase, pancreatin and leucozyme; or trypsin, pancreatin and leucozyme.

In one embodiment, the variable domain is displayed on bacteriophage when incubated under condition (i) or (ii) for example at a phage library size of 10⁶ to 10¹³ eg 10⁸ to 10¹² replicative units (infective virions).

In one embodiment, the variable domain specifically binds its target following incubation under condition (i) or (ii), eg assessed using BiaCore™ or ELISA, eg phage ELISA or monoclonal phage ELISA.

In one embodiment, the variable domains of the invention specifically bind protein A or protein L. In one embodiment, specific binding to protein A or L is present following incubation under condition (i) or (ii).

In one embodiment, the variable domains of the invention may have an OD₄₅₀ reading in ELISA, eg phage ELISA or monoclonal phage ELISA) of at least 0.404, eg, following incubation under condition (i) or (ii).

In one embodiment, the variable domains of the invention display (substantially) a single band in gel electrophoresis, eg following incubation under condition (i) or (ii).

The compositions described herein comprise (a) a polypeptide e.g. a domain antibody (dAb), and (b) a physiologically acceptable buffer, e.g. one which has a pH range of between about 4 and about 8 and a viscosity which is about equal to the viscosity of a solution of about 2% to about 10% PEG 1000 in 50 mM phosphate buffer containing 1.2% (w/v) sucrose; and wherein the composition comprises liquid droplets and about 40% or more, e.g. 50% or more, of the liquid droplets present in the composition have a size which is less than about 6 microns e.g. in the range from about 1 to about 6 microns e.g. less than about 5 microns e.g. from about 1 or about 2 to about 5 microns. For deep lung delivery it can be useful to have particles sizes ranging from about 1 to about 3 microns.

Suitable buffers which can be utilised according to the invention are physiologically acceptable buffers e.g. those which can be safely administered to the lung, for example phosphate, citrate, acetate or histidine buffers.

pH range: In one embodiment the pH of the buffer is in the range of from about 4 to about 8, e.g. about 7 to about 7.5, or from about 5 to about 6.

In certain embodiments the viscosity of the buffer is preferably about equal to the viscosity of a solution of about 2% to about 10% PEG 1000 in 50 mM phosphate buffer containing 1.2% (w/v) sucrose. Viscosity can be measured using standard techniques know in the art such as the cone and plate method, or the magnetic bead microrheometer method.

The size of the liquid droplets present in the composition can be measured using standard methods for measuring particle size e.g. using a Malvern laser scanning device or e.g. using Cascade Impactors such as the Dekatie Impactor Device or the Marple Impactor Device.

Useful additives which can be added to the buffer e.g. to effect a change in viscosity can be for example Polyethylene glycol (PEG) such as PEG 1000 or sugars e.g. sucrose, mannose, other additives can comprise e.g. stabilising agents such as detergents e.g. Tweens.

A polypeptide, such as a dAb, for formulation for direct pulmonary delivery as described herein can usefully be for example an anti-TNFR1 binder e.g. a dAb which binds anti-TNFR1, and for example it can be a substantially antagonistic anti-TNFR1 dAb such as an antagonistic anti-TNFR1 dAb as disclosed in WO 2007/049017 (the contents and teachings of which are specifically incorporated herein by reference), e.g. it can have an encoding sequence as described in any one of SEQ ID 1-650 of WO 2007/049017, all of which sequences are specifically incorporated herein by reference. Such anti-TNFR1 dAb compositions can be useful therapeutics for treating respiratory diseases, e.g. COPD and asthma.

It can also be desirable for the polypeptides of the compositions e.g. the dAbs, to have a high melting temperatures (Tm). It has for example been shown that dAbs with higher melting temperatures (Tms) are more resistant to aggregation induced by shear stress, elevated temperatures and long term storage stability. Both shear and thermal stress play a significant role in inducing aggregate formation during nebulisation and so it is advantageous to select for dAbs with high Tm's. One method of achieving this would be to produce a phage display library of the dAb and use a protease (such as trypsin) to select and identify molecules which are resistant to the action of the enzyme. The protease resistant dAbs generated by this method have a higher melting temperature and this has been attributed to changes in the sequence of the dAb which increase the core stability of the protein so that the peptide backbone of the molecule is less accessible to hydrolysis by the enzyme.

Protease resistant domain antibodies and methods for selecting them are for example further described in U.S. Ser. No. 60/933,632 (the teachings of which are herein incorporated by reference).

The improvement in the Tm of the dAbs could also be achieved for example by heating the dAbs during the selection process. Hence when the compositions of the invention comprise domain antibodies it can be desirable for the dAbs to have a Tm in the range from about e.g. 55 deg C. to about e.g. 90 deg C. e.g. from about 80 deg C. to about e.g. 90 deg C. The Tm can be determined using standard techniques such as differential scanning calorimetry (DFC).

The polypeptide concentration e.g. a dAb which can be used in the compositions described herein can range from about 1 mg/ml up to about 40 mg/ml. Higher concentrations e.g. of dAbs, e.g. about 20 mg/ml to about 40 mg/ml, can be useful for improving delivery to the lung.

The invention is further described by way of illustration only in the following examples:

### EXAMPLES

Lead Selection & Characterisation of domain antibodies to human TNFR1 is described in detail below:

Domain antibodies generated were derived from Domantis' phage libraries. Both soluble selections and panning to passively absorbed human TNFR1 were performed according to the relevant standard Domantis methods. Human TNFR1 was purchased as a soluble recombinant protein either from R&D systems (Cat No 636—R1-025/CF) or Peprotech (Cat no. 310-07) and either used directly (in the case of passive selections) or after biotinylation using coupling via primary amines followed by quality control of its activity in a biological assay and analysis of its MW and extent of biotinylation by mass spectrometry. Typically 3 rounds of selection were performed utilising decreasing levels of antigen in every next round.

Outputs from selections were screened by phage ELISA for the presence of anti-TNFR1 binding clones. DNA was isolated from these phage selections and subcloned into a expression vector for expression of soluble dAb fragments. Soluble dAb fragments were expressed in 96-well plates and the supernantants were used to screen for the presence of anti-TNFR1 binding dAbs, either using a direct binding ELISA with anti-c-myc detection or BIAcore™ using a streptavidin/biotinylated TNFR1 BIAcore™ chip and ranked according to off-rates.

The lead molecules, described below, were derived from the parental dAb, designated DOM1h-131. This molecule was selected from the phage display library after 3 rounds of selections using 60 nM of biotinylated antigen. Streptavidin or neutravidin coated Dyna beads were alternated as capture reagents in each round of selection to prevent selection of binders against either streptavidin or neutravidin. The potency of the lead DOM1h-131 at this stage was in the low micromolar range as determined in the MRC-5 fibroblast/IL-8 release cell assay. The binding kinetics as determined by BIAcore™ typically displayed fast-on/fast-off rates. E. coli expression levels of this DOM1h-131 lead molecule, as a C-terminally myc tagged monomer were in the region of 8 mg/l.

### Affinity Maturation of Leads:

DOM1 h-131 was taken forward into affinity maturation to generate mutants with higher potency and improved biophysical characteristics (see FIG. 1 for amino acid sequences of DOM1h-131 derived leads). After generation of an error-prone library (average number of 1 amino acid change per dAb sequence, library size 8×10⁷) using an error-prone PCR polymerase (Genemorph II, Stratagene), seven rounds of selection utilising these error-prone libraries were performed. This strategy led to the isolation of clone DOM1h-131-8, a molecule where 4 amino acid changes (one in framework 1 (FR1), one in CDR1, one in CDR3 and one in FR4) gave an approximate 100-fold improvement in potency as measured by the MRC-5 assay (˜4 nM).

In order to further improve potency, single amino acid positions were diversified by oligo-directed mutagenesis at key positions suggested by the error-prone lead consensus information. During this process an improved version of the DOM1h-131-8 clone, DOM1h-131-24 (originally named DOM1h-131-8-2 prior to correction) was isolated through BIAcore™ screening that had a single K94R amino acid mutation (amino acid numbering according to Kabat) and an RBA potency of 200-300 pM.

Further error-prone libraries based on this lead and the NNS library from which it was derived were generated and subjected to three rounds of phage selections using heat treatment (for method see Jespers L, et al., Aggregation-resistant domain antibodies selected on phage by heat denaturation. Nat. Biotechnol. 2004 September; 22(9):1161-5). During this selection, libraries were pooled and clones derived from round two of the selection yielded dAbs such as DOM1h-131-53 which were considered to be more heat stable. It was hypothesised that these clones would possess better biophysical characteristics. Some framework mutations in clone DOM1h-131-53 were germlined to generate clone DOM1h-131-83. This clone formed the basis for further diversification via oligo-directed individual CDR mutagenesis either using phage display selection as described above or using the in-vitro compartmentalization technology using emulsions. The phage display strategy generated leads DOM1h-131-117 and DOM1h-131-151. The in-vitro compartmentalization technology generated DOM1h-131-511.

At this stage these three leads were compared in biophysical and biological assays and DOM1h-131-511 was the molecule with the best properties. Furthermore these molecules were tested for their resistance to proteolytic cleavage in the presence of trypsin or leucozyme. Leucozyme consists of pooled sputum from patients with cystic fibrosis and contains high levels of elastase and other proteases and was used as a surrogate for in vivo conditions in lung diseases. This data indicated that all three leads DOM1h-131-117, DOM1h-131-151 and DOM1h-131-511 were rapidly degraded in presence of trypsin or leucozyme. This finding raised concerns about the in vivo persistence of DOM1h-131-511 when in the patient and a strategy was developed to select for improved resistance to trypsin. It was hypothesised that such improved trypsin resistance could have a beneficial effect on other biophysical properties of the molecule. Essentially the standard phage selection method was modified to allow for selection in the presence of proteases prior to selection on antigen. To this end a new phage vector was engineered in which the c-myc tag was deleted to allow selections in the presence of trypsin without cleaving the displayed dAb off the phage. DOM1h-131-511 based error-prone libraries were generated and cloned in the new pDOM33 vector. Phage stocks generated from this library were pre-treated with trypsin, subsequently protease inhibitor was added to block the trypsin activity prior to selection on the relevant antigen. Four rounds of selection were performed. Soluble expressed TNFR1 binding dAbs were assessed using the BIAcore™ for their ability to bind TNFR1 with or without the presence of proteases. This led to the isolation of two lead molecules DOM1h-131-202 and DOM1h-131-206 which demonstrated improved protease resistance as shown by BIAcore™ antigen binding experiments. It is interesting to note that DOM1h-131-202 contained only one mutation in CDR2 (V53D, all amino acid numbering according to Kabat) in comparison to DOM1h-131-511, whereas DOM1 h-131-206 contained only two mutations: the first mutation is the same as in DOM1h-131-202 (V53D mutation in CDR2) and the second is a Y91H mutation in FR3 (see FIG. 1). This Y91H mutation in FR3 does occur in the 3-20 human germline gene indicating that this residue occurs in human antibodies. The three clones DOM1h-131-511, DOM1h-131-202 and DOM1h-131-206 have amino acid sequences as shown in FIG. 1.

### Activity of the Molecules was Determined as Below:

BIAcore™ binding affinity assessment of DOM1H-131-202, DOM1H-131-511 and DOM1H-131-206 for binding to human TNFR1.

The binding affinities of DOM1H-131-202, DOM1H-131-511 and DOM1H-131-206 for binding to human recombinant E. coli expressed human TNFR1 were assessed by BIAcore™ analysis. Analysis was carried out using biotinylated human TNFR1. 1400 RU of biotinylated TNFR1 was coated to a streptavidin (SA) chip. The surface was regenerated back to baseline using mild acid elution conditions. DOM1H-131-202, DOM1H-131-511 and DOM 1H-131-206 were passed over this surface at defined concentrations using a flow rate of 50 μl/min. The work was carried out on a BIAcore™ 3000 machine and data were analysed and fitted to the 1:1 model of binding. The binding data fitted well to the 1:1 model for all tested molecules. All K_{D} values were calculated from kₒₙ and k_{off} rates. BIAcore™ runs were carried out at 25° C.

The data below were produced from three independent experiments. In each experiment the results were calculated by averaging a number of fits using highest dAb concentrations for kd and lower concentrations for ka. The data are presented as the mean and standard deviation (in brackets) of the results (Table 1).

TABLE 1
BIAcore ™ data for DOM1H-131-202, DOM1H-131-511 and DOM1H-
131-206 binding to human TNFR1

| | kₒₙ | k_{off} | K_{D} (nM) |
|---|---|---|---|
| DOM1H-131-511 | 5.03E+05 | 5.06E−04 | 1.07 |
| (511) | (1.07E+05) | (1.01E−04) | (0.44) |
| DOM1H-131-202 | 1.02E+06 | 5.42E−04 | 0.55 |
| (202) | (2.69E+05) | (3.69E−05) | (0.11) |
| DOM1H-131-206 | 1.55E+06 | 7.25E−04 | 0.47 |
| (206) | (3.57E+05) | (1.95E−04) | (0.06) |

DOM1H-131-202, DOM1H-131-511 and DOM1H-131-206 bound similarly and with high affinity to human TNFR1. DOM114-131-202 and DOM1H-131-206 bind with average affinities of 0.55 nM and 0.47 nM respectively. Both DOM1H-131-202 and

DOM1H-131-206 have a slightly better affinity in comparison to DOM1H-131-511 which has an average affinity of 1.07 nM.

### Receptor Binding Assay:

The potency of the dAbs was determined against human TNFR1 in a receptor binding assay. This assay measures the binding of TNF-alpha to TNFR1 and the ability of soluble dAb to block this interaction. The TNFR1 is captured on a bead pre-coated with goat anti-human IgG (H&L). The receptor coated beads are incubated with TNF-alpha (10 ng/ml), dAb, biotin conjugated anti-TNF-alpha and streptavidin alexa fluor 647 in a black sided clear bottomed 384 well plate. After 6 hours the plate is read on the ABI 8200 Cellular Detection system and bead associated fluorescence determined. If the dAb blocks TNF-alpha binding to TNFR1 the fluorescent intensity will be reduced.

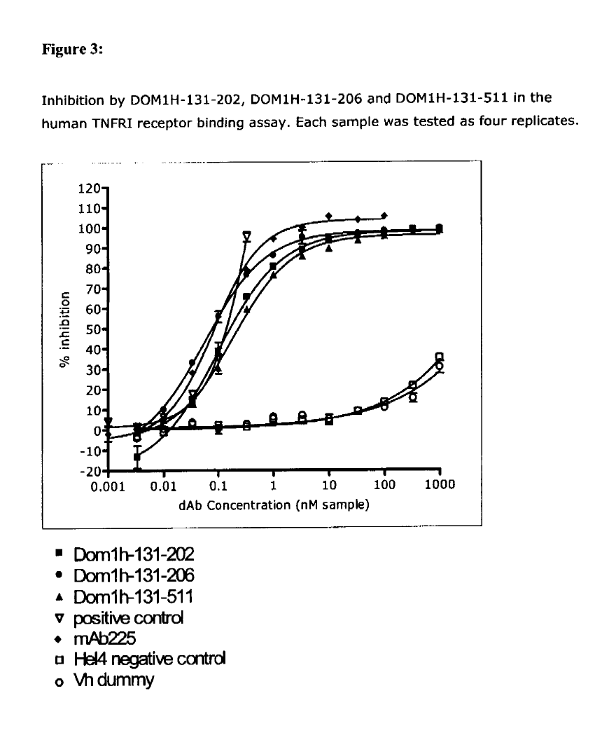

Data was analysed using the ABI 8200 analysis software. Concentration effect curves and potency (EC₅₀) values were determined using GraphPad Prism and a sigmoidal dose response curve with variable slope. The assay was repeated on three separate occasions. A TNF-alpha dose curve was included in each experiment (FIGS. 2 and 3). The concentration of TNF-alpha used to compete with dAb binding to TNFR1 (10 ng/ml) is approximately 90% of the maximum TNF-alpha response in this assay.

A representative graph is shown in FIG. 3 showing the ability of dAbs to inhibit the binding of TNF-alpha to TNFR1. In all three experiments the negative control samples (HEL4 and V_{H} dummy) weakly inhibit the interaction between TNF-alpha and TNFR1 at high concentrations. The average potency (EC₅₀) values for the test samples and positive controls (anti-TNFR1 mAb obtained from R&D Systems, mAb225) and Enbrel™ (etanercept; a dimeric fusion consisting of TNFR2 linked to the Fc portion of IgG1; licensed for the treatment of rheumatoid arthritis) are shown in Table 2.

TABLE 2
Potency (EC₅₀) values for DOM1H-131-202, DOM1H-131-206 and
DOM1H-131-511 in a TNFR1 receptor binding assay for three
repeat experiments.

| Sample | Average EC₅₀ (nM) | SEM |
|---|---|---|
| DOM1H-131-202 | 0.11 | 0.008 |
| DOM1H-131-206 | 0.07 | 0.01 |
| DOM1H-131-511 | 0.19 | 0.01 |
| Enbrel ™ (Etanercept) | 0.20 | 0.07 |
| Anti-TNFR1 mAb # mAb225 | 0.08 | 0.003 |

In this assay DOM1H-131-206 appears more potent than the other two dAbs being tested and has a similar potency to the commercially available anti-TNFR1 mAb, MAB225 (R and D Systems).

Expression of Lead Clones from Pichia pastoris was Carried Out as Described Below:

The primary amino acid sequence of the three lead molecules was used to produce codon optimised genes for secreted expression in Pichia pastoris. The three synthetic genes were cloned into the expression vector pPIC-Zα (from Invitrogen) and then transformed into two Pichia strains, X33 and KM71H. The transformed cells were plated out onto increasing concentrations of Zeocin (100, 300, 600 and 900 μg/ml) to select for clones with multiple integrants. Approximately 15 clones for each cell line and construct were selected for expression screening. As the correlation between high/low gene copy number and expression level is not fully understood in Pichia pastoris, several clones were picked from across the Zeocin concentration range. 5 L fermenter runs were carried out using clones that had not been extensively screened for high productivity. This allowed the production of significant amounts of material for further studies.

### Material Production for Protein Characterisation:

Protein A based chromatography resins have been extensively used to purify V_{H} dAbs from microbial culture supernatants. Although this allows a single step purification method for producing high purity material, usually >90% in most cases, for some molecules the low pH elution conditions can result in the formation of aggregates. There is also the issue of the limited capacity of affinity resins for dAbs; this would mean the use of significant quantities of resin to process from fermenters. In order to produce high quality material for characterisation and further stability and nebuliser studies, a downstream purification process was devised using a mixed modal charge induction resin as the primary capture step followed by anion exchange. Without significant optimisation, this allowed the recovery of ˜70% of the expressed dAb at a purity of ˜95%. For the capture step on the mixed modal charge induction resin, Capto MMC from GE Healthcare, column equilibration is performed using 50 mM sodium phosphate pH6.0 and the supernatant is loaded without any need for dilution or pH adjustment. After washing the column, the protein is eluted by pH gradient using an elution buffer which is 50 mM Tris pH 9.0. The specific wash and gradient conditions will vary slightly depending on the pI of the protein being eluted.

The eluate peak is then further purified with a flow through step using anion exchange chromatography. This removes residual HMW contamination such as alcohol oxidase and reduces endotoxin. The resin is equilibrated with either PBS or phosphate buffer pH 7.4 without salt. Upon loading the eluate from Capto MMC onto the anion exchange resin the dAb does not bind and is recovered from the flow through. Endotoxin and other contaminants bind to the resin. The presence of salt if using PBS buffer improves protein recovery to 91% for this step rather than 86% recovery achieved without salt. However the presence of salt reduces the effectiveness of endotoxin removal such that a typical endotoxin level of dAb following this step with the inclusion of salt was measured as 58 EU/ml compared with a level of <1.0 EU/ml obtained when no salt was present.

### Protein Characterisation:

The material produced from the 5 L fermenter runs was characterised for identity using electrospray mass spectrometry, amino terminal sequencing and isoelectric focusing and for purity using SDS-PAGE, SEC and Gelcode glycoprotein staining kit (Pierce).

### Identity:

The amino terminal sequence analysis of the first five residues of each protein, was as expected (EVQLL . . . ). Mass spectrometry was performed on samples of the proteins which had been buffer exchanged into 50:50 H₂O:acetonitrile containing 0.1% glacial acetic acid using C4 Zip-tips (Millipore). The measured mass for each of the three proteins was within 0.5Da of the theoretical mass based on the primary amino acid sequence (calculated using average masses) when allowing for a mass difference of −2 from the formation of the internal disulphide bond. IEF was used to identify the proteins based on their p1 which was different for each protein.

### Purity:

The three proteins were loaded onto non-reducing SDS-PAGE gels in 1 μg and 10 μg amounts in duplicate. A single band was observed in all instance.

Size exclusion chromatography was also performed to demonstrate levels of purity. For size exclusion chromatography (SEC) 100 μg of each protein were loaded onto a TOSOH G2000 SWXL column flowing at 0.5 ml/min. Mobile phase was PBS/10% ethanol.

### Investigation of Dab Stability for Candidate Selection:

For the indication of COPD, it would be necessary to deliver the dAb into the lung using a nebuliser device. This would mean the protein could potentially experience a range of shear and thermal stresses depending on the type of nebuliser used and could be subjected to enzymatic degradation by proteases in the lung environment. It was clearly essential to know if the protein could be delivered using this type of device, form the correct particle size distribution and remain functional following nebuliser delivery. Therefore the intrinsic stability of each molecule to a range of physical stresses was investigated to determine the baseline stability and the most sensitive stability indicating assays. As the stability of each protein will be dependent on the buffer solution it is solubilised in, some pre-formulation work was necessary. This information, such as buffer, pH, would also be useful for understanding the stability of the protein during the downstream purification process and subsequent storage. In order to characterise the changes in the molecules during exposure to a range of physical stresses, a range of analytical techniques were used such as size exclusion chromatography (SEC), SDS-PAGE and isoelectric focusing (IEF).

### Assessment of Protease Stability of DOM1H-131-202, DOM1H-131-511 and DOM1H-131-206:

The protease stability of DOM1H-131-202, DOM1H-131-511 and DOM1H-131-206 was assessed by BIAcore™ analysis of the residual binding activity after pre-incubation for defined timepoints in excess of proteases. Approximately 1400RU of biotinylated TNFR1 was coated to a streptavidin (SA) chip. 250 nM of DOM1H-131-202, DOM1H-131-511 and DOM1H-131-206 was incubated with PBS only or with 100 ug/ml of trypsin, elastase or leucozyme for 1, 3, and 24 hours at 30° C. The reaction was stopped by the addition of a cocktail of protease inhibitors. The dAb/protease mixtures were then passed over the TNFR1 coated chip using reference cell subtraction. The chip surface was regenerated with 10 ul 0.1M glycine pH 2.2 between each injection cycle. The fraction of DOM1H-131-202, DOM1H-131-511 and DOM1H-131-206 bound to human TNFR1 (at 10 secs) pre-incubated with proteases was determined relative to dAb binding without proteases. BIAcore™ runs were carried out at 25° C.

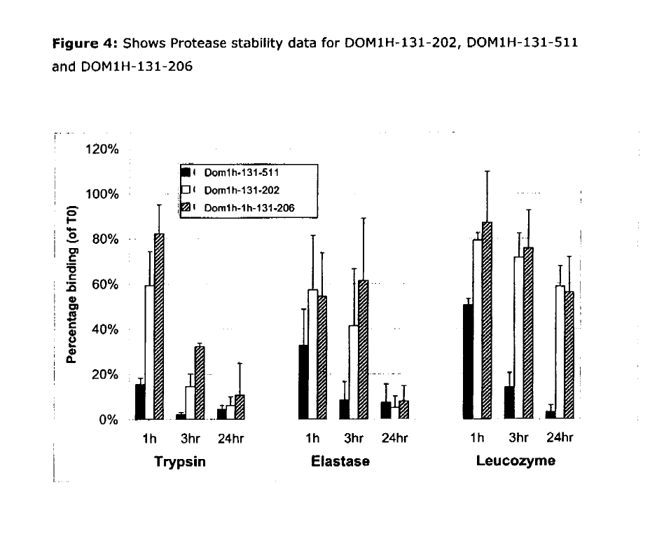

The data was produced from three independent experiments. The bar graph indicates mean values and the error bars indicate standard deviation of the results (for results see FIG. 4).

It was found that DOM1H-131-202 and DOM1H-131-206 were shown to have greater resistance to proteolytic degradation by trypsin, elastase or leucozyme in comparison to DOM1H-131-511. The difference between DOM1H-131-202 and DOM1H-131-206 in comparison to DOM1H-131-511 is most pronounced after 1 hr with trypsin and after 3 hrs with elastase or leucozyme.

### Thermal Stability as Determined Using DSC:

In order to determine at which pH the molecules had the greatest stability, differential scanning calorimeter (DSC) was used to measure the melting temperatures (Tₘ) of each dAb in Britton-Robinson buffer. As Britton-Robinson is made up of three component buffer systems (acetate, phosphate and borate), it is possible to produce a pH range from 3-10 in the same solution. The theoretical pI was determined from the proteins primary amino acid sequence. From the DSC, the pH at which the dAbs had their greatest intrinsic thermal stability was found to be pH 7 for DOM1H-131-202 (202), pH 7-7.5 for DOM1H-131-206 (206) and pH 7.5 for DOM1H-131-511 (511). For all subsequent stress and stability work the following pHs were used for each dAb; for DOM1H-131-202 (202) and DOM1H-131-206 (206) pH 7.0 and for DOM1H-131-511 (511) pH 7.5 in Britton-Robinson buffer. The results are summarised in Table 3.

TABLE 3
Summary of the pH and Tₘ^{s} of DOM1H-131-202 (202), DOM1H-131-
206 (206) and DOM1H-131-511 (511) as determined by DSC in
Britton-Robinson buffer at 1 mg/ml

| | pH that gives greatest | |
|---|---|---|
| | intrinsic thermal | Tm (° C.) of the dAb |
| dAb | stability | at the given pH |
| DOM1H-131-202 (202) | 7.0 | 68.6 |
| DOM1H-131-206 (206) | 7.0-7.5 | 65.8 |
| DOM1H-131-511 (511) | 7.5 | 58.0 |

### Intrinsic Solubility Testing:

All the lead dAbs were concentrated in centrifugal Vivaspin concentrators (5K cut-off), to determine their maximum solubility and the levels of recovery upon concentration. Experiments were performed in Britton-Robinson buffer at the most stable pH. Sample volumes and concentrations were measured over a time course and deviation from expected concentration recorded as well as percent recovery of the sample.

It was found that all proteins could be concentrated to over 100 mg/ml in Britton-Robinson buffer. Both DOM1H-131-202 (202) and DOM1H-131-206 (206) showed lower recoveries than expected compared to DOM1H-131-511 (511), but still within acceptable levels.

Nebuliser Delivery of the Lead dAbs:

By testing different nebulisers and formulation buffers it was demonstrated that the dAb could effectively be delivered using a wide range of nebulising devices. More importantly, it was shown for the first time that nebulisation of the dAb in the formulation buffer produced the preferred particle size distribution (compared using the percentage of droplets <5 μm) for effective lung delivery whilst maintaining protein functionality. This is further described below.

### Comparison of Performance in Various Devices:

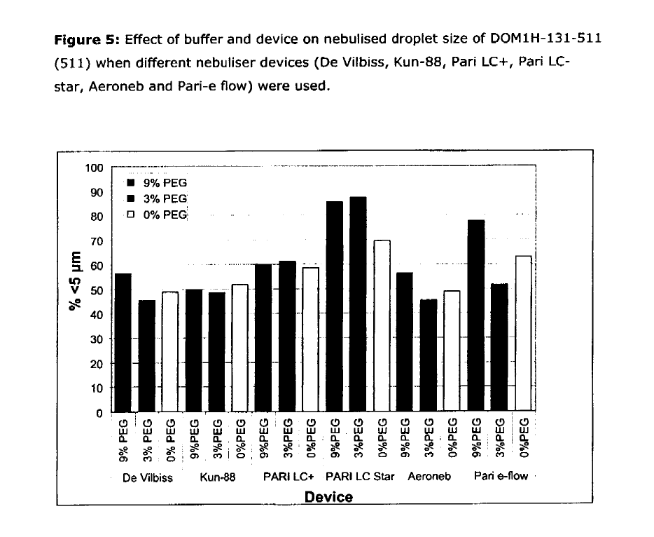

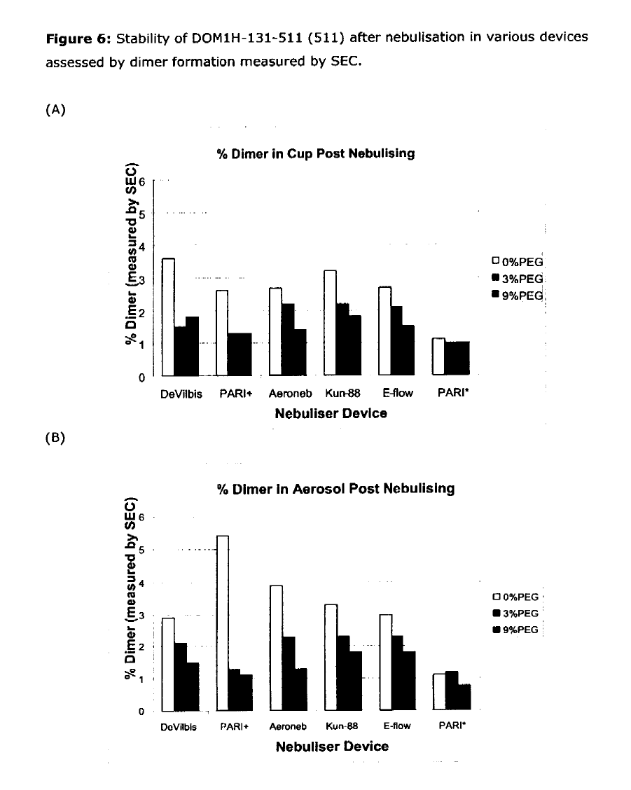

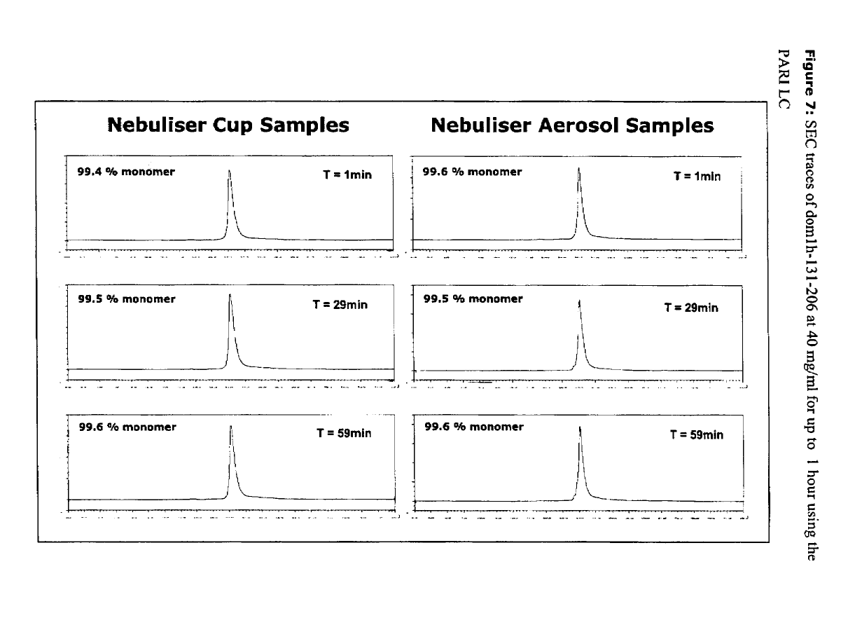

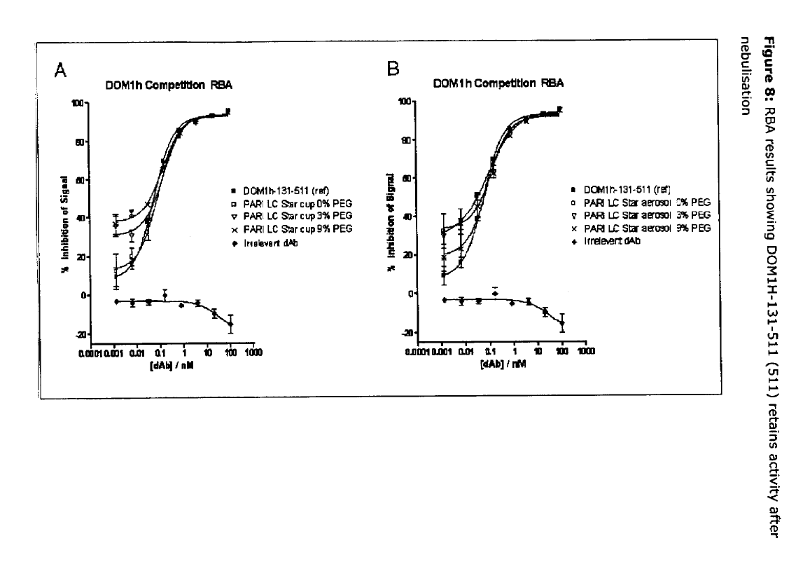

DOM1H-131-511 (511) was tested in six nebuliser devices comprising two devices from each of the three main groups of nebulisers for liquid formulations i.e. ultrasonic nebulisers, jet nebulisers and vibrating mesh nebulisers. In each device the dAb was tested at 5 mg/ml with a range of PEG concentrations. For each sample the percentage of droplet size <5 μm was measured using a Malvern Spraytek Device (Malvern Instruments Limited, UK) and the results are shown in FIG. 5. The stability of each sample after being nebulised was assessed using SEC to analyse the amount of sample which had dimerised both in the material remaining in the cup and in collected aerosol. The results may be seen in FIG. 6. The less the extent of dimer formation the greater the stability. FIG. 7 also shows SEC traces that demonstrate that even at 40 mg/ml in PBS GSK 206 remains stable to nebulisation and there is low dimmer formation.

Most devices can deliver 40% or more of the liquid formulation in the correct size range but the eFlow (a vibrating mesh nebuliser device) and PARI LC (a jet nebuliser) devices perform better, with the PARI LC* (star) device delivering more than 80% when PEG is included in the buffer. This increase in delivery with PEG is also observed with the eFlow and, to a lesser extent, with the PARI LC+. Importantly activity of the dAb was also found to be retained after nebulisation (see results in FIG. 8)

### Effect of Buffer Additives:

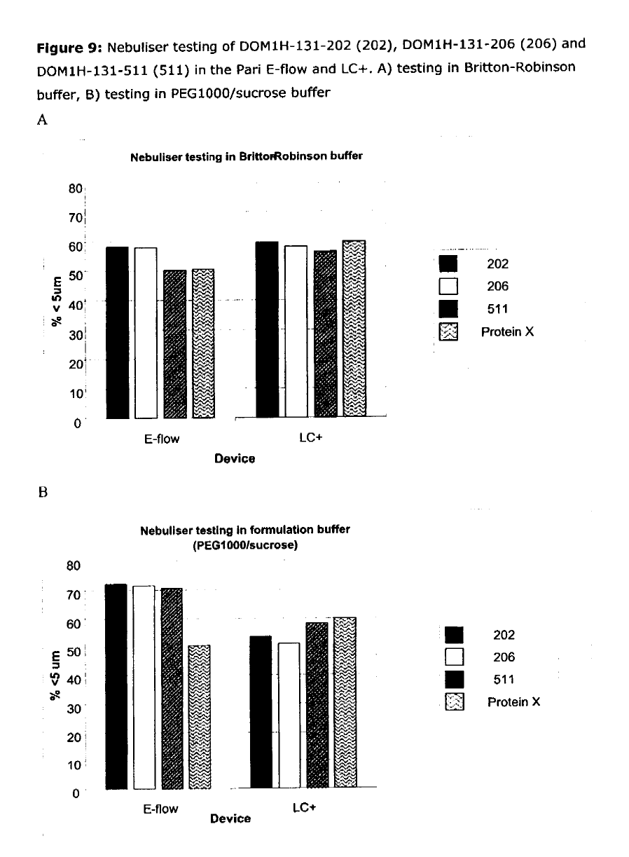

Due to the lower stability of DOM1H-131-511 (511), the 50 mM phosphate formulation buffer contained both PEG 1000 and sucrose (and has a viscosity which is within the range which is defined as about equal to the viscosity of a solution of about 2% to about 10% PEG 1000 in 50 mM phosphate buffer containing 1.2% (w/v sucrose) to help protect the dAb from both shear and thermal stress. As both DOM1H-131-202 (202) and DOM1H-131-206 (206) have higher Tₘ's and showed considerably improved stability to thermal stress, all the molecules were tested in both the original formulation buffer and in Britton-Robinson buffer (which has a lower viscosity than the formulation buffer). The dAbs were tested in both the E-flow and Pari LC+devices with run time of 3.5 minutes at a protein concentration of 5 mg/ml and the particle size distribution determined using a Malvern Spraytek Device. As a comparison, a marketed drug for cystic fiborosis (designated standard protein X) that is delivered using a nebuliser device, was tested in its own formulation buffer. The results are shown in FIG. 9. For good delivery and distribution into the deep lung, the ideal particle size is less than 6 microns, e.g. <5 μm. All the dAbs give comparable levels of particle sizes that were less than 5 μm in both the Britton-Robinson buffer and formulation buffer (as described earlier). However, the higher viscosity of the formulation buffer could be particularly beneficial for producing particles within the correct size range, e.g. particles <5 μm.

The concentration of the dAb in the cup of the device was determined by A₂₈₀ measurements before and after nebulisation. It was found that the protein concentration did not change significantly indicating that neither the protein nor vehicle is preferentially nebulised during delivery.

Nebuliser delivery of Anti-IL13 Monomeric dAbs:

It could also be desirable for dAbs to be delivered directly to the site of action for other pulmonary conditions such as asthma. By testing monomeric dAbs which bind the cytokine IL13 it was demonstrated that dAbs could be administered effectively by pulmonary delivery using a nebuliser and could potentially be used for the treatment of asthma. This is further described below.

dAbs that bind IL13 have been described WO 2007/085815 and of the dAbs listed therein, two were selected to be tested for nebuliser delivery based on their potency against the target and their biophysical properties—these were DOM10-53-474 and DOM10-275-78.

Potencies of Anti-IL-13 dAbs DOM10-53-474 and DOM10-275-78

### In a HEK Cell Assay:

This assay uses HEK293 cells stably transfected with the STAT6 gene and the SEAP (secreted embryonic alkaline phosphatase) reporter gene (Invitrogen, San Diego). Upon stimulation with IL-13 SEAP is secreted into the supernatant which is measured using a colorimetric method. Soluble dAbs were tested for their ability to block IL-13 signalling via the STAT6 pathway. Briefly, the dAb is pre-incubated with 6 ng/ml recombinant IL-13 (GSK) for one hour then added to 50000 HEKSTAT6 cells in DMEM (Gibco, Invitrogen Ltd, Paisley, UK) in a tissue culture microtitre plate. The plate is incubated for 24 hours at 37° C. 5% CO₂. The culture supernatant is then mixed with QuantiBlue (Invivogen) and the absorbance read at 640 nm. Anti-IL-13 dAb activity causes a decrease in STAT6 activation and a corresponding decrease in A₆₄₀ compared to IL-13 stimulation.

### Table 4:

The Results Summary Table 4 below shows the EC50 of each of the selected dAbs against both human IL-13 (hIL-13) and cyno IL-13 (cIL-13):

TABLE 4

| | 10-53-474 EC50 | 10-275-78 EC50 |
|---|---|---|
| | (nM) | (nM) |
| HEK assay hIL-13 | 0.63 (n = 13) | 2.5 (n = 7) |
| HEK assay cIL-13 | 11.1 (n = 10) | 1.4 (n = 7) |

DOM10-53-474 is the preferred clinical candidate owing to its superior potency, however it would have reduced potency in a NHP clinical model. DOM10-275-78 would have improved potency in a NHP clinical model.

### DOM10 Sandwich ELISA:

This assay also measures the potency of the dAbs. This assay uses a mouse anti-human IL-13 capture antibody and a separate mouse anti-human IL-13 detection antibody (bender MedSystems, cat no BMS231/3MST). The capture Ab is captured on an ELISA plate, then a mixture of IL-13 (GSK) and dAb protein is added. The captured IL-13 is then detected using a biotinylated anti-IL-13 detection Ab and streptavidin-HRP. The plate is developed using a colorimetric substrate and the OD read at 450 nm. dAb blocking IL-13 binding is shown by a decrease in OD.

TABLE 5
Results Summary Table 5 showing the EC50 of each of the selected
dAbs against human IL-13

| | 10-53-474 EC50 | 10-275-78 EC50 |
|---|---|---|
| | (nM) | (nM) |
| Sandwich assay | 0.057 (n = 3) | 3.326 (n = 3) |
| hIL-13 | | |

### DOM10 Binding Assay:

This assay uses biotinylated human IL-13 (biotinylated GSK IL-13 produced in-house) to capture the dAb. An ELISA plate is coated with NeutrAvidin (Pierce, cat no. 31000) to capture biotinylated IL-13. The dAb is added and bound dAb is detected using a rabbit anti-human Ig (VH specific) Ab and then a HRP conjugated anti-rabbit IgGAM Ab (Sigma Cat A-2074). The plate is developed using a colorimetric substrate and the OD read at 450 nm. The signal from the assay is proportional to the amount of dAb bound.

TABLE 6
Results Summary Table 6 showing the EC50 of each of the selected dAbs

| | 10-53-474 EC50 | 10-275-78 EC50 |
|---|---|---|
| | (nM) | (nM) |
| Binding assay | 1.08 (n = 5) | 1.304 (n = 10) |

### BIACORE® Off-Rate Screening:

A streptavidin coated SA chip (Biacore) was coated with approximately 100 RU of biotinylated human IL-13 (R&D Systems, Minneapolis, USA) or cynomolgous IL-13 (Produced in-house). dAbs were serially diluted in HBS-EP running buffer. 50 to 100 ul of the diluted supernatant was injected (kininject) at 50 ul/min flow rate, followed by a 5 minute dissociation phase. Association and dissociation off-rates and constants were calculated using BIAevaluation software v4.1 (Biacore).

TABLE 7
comparing the binding affinities of the two selected dAbs DOM10-53-474
and DOM10-275-78 for both human and cyno IL-13.

| | DOM10-53-474 | DOM10-275-78 |
|---|---|---|
| | (nM) | (nM) |
| Biacore hIL-13 | 0.028 | 0.072-0.1 |
| Biacore cIL-13 | 2.0 | 0.32-0.75 |

### Binding to Variant IL-13 (R1300):

Genetic variants of IL-13, of which R130Q is a common variant, have been associated with an increased risk for asthma (Heinzmann et al. Hum Mol. Genet. (2000) 9549-59) and bronchial hyperresponsiveness (Howard et al., Am. J. Resp. Cell Molec. Biol. (2001) 377-384). Therefore it is desirable for the anti IL-13 dAb to also have binding affinity for this variant of the cytokine. DOM10-53-474 bound IL-13 (R130Ω) and inhibited IL-13 (R130Q) stimulated proliferation in two cell assay (TF-1 & Hek-Stat6).

TABLE 8
This shows binding affinity of DOM10-53-474 for variant IL-13

| Cell Assay | EC50 nM |
|---|---|
| Hek-Stat6 (variant hIL-13 stimulation = | 0.273 (n = 4) |
| 3 ng/ml) | |
| TF-1 (variant hIL-13 stimulation = | 0.133 (n = 3) |
| 5 ng/ml) | |

### Agonistic Activity:

To determine whether DOM10-53-474 binds non-target proteins, and to ensure that no undesired cytokines/interferons are released due to agonistic activity of the dAb, DOM10-53-474 was tested for agonistic activity in a human blood assay. Each sample was titrated from 1 μM to 10 nM of DOM10-53-474 and tested in two donors, A & B. The assay was set up in duplicate (a & b) and the meso scale discovery (MSD) was performed in duplicate. The nil wells contained blood alone, (i.e. no dAb added), there were 8 nil wells for donor A and 4 for donor B. The cytokines assayed were IL-8, IL-6, TNFα, IL-10, IL-1f3, IL-12p70 and IFNγ. No agonistic activity was seen with respect to IL-6, TNFα, IL-10, IL-113, IL-12p70 or IFNγ. There was a little IL-8 production at the 1 μM concentration but this was very low.

### SEC-MALLS:

The in-solution properties of dAb proteins were determined by an initial separation on SEC (size exclusion chromatography; TSKgel G2000/3000SWXL, Tosoh Biosciences, Germany; BioSep-SEC-S2000/3000, Phenomenex, Calif., USA) and subsequent on-line detection of eluting proteinaceous material by UV (Abs280 nm), R1 (refractive index) and light scattering (laser at 685 nm). The proteins were at an initial concentration of 2 mg/mL for DOM10-275-78 and 1.4 mg/ml for DOM10-53-474, as determined by absorbance at 280 nm, and visually inspected for impurities by SDS-PAGE. The homogeneity of samples to be injected was usually >90%. 100 uL were injected onto the SEC column. The protein separation on SEC was performed at 0.5 mL/min for 45 minutes. PBS (phosphate buffered saline ±10% EtOH) was used as mobile phase. The ASTRA software (Wyatt Inc; CA; USA) integrated the signals of all three detectors and allowed for the determination of the molar masses in kDa of proteins from ‘first physical principles’. Inter-run variations and data quality was assessed by running a positive control of known in-solution state with every sample batch.

For some DOM10-53 clones no reliable solution state could be assigned because the molecules bound aspecifically to the column matrix or could not be resolved using the size exclusion column. For these cases where the solution state was reliable (i.e. DOM10-53-474 and DOM10-275-78) it was demonstrated that the DOM10-275-78 molecule is mostly a monomer in solution and 90% is eluted from the

column and that for the DOM10-53-474 molecule the majority of the protein is clear monomer. DOM10-53-474 eluted as a single peak with the molar mass defined as 13 kDa in the right part of the peak (monomer) but creeping up over the left part of the peak up to 18 kDa, indicating some degree of rapid self association (average mass over this peak is 14 kDa).

### Differential Scanning Calorimetry (DSC):

DOM10-275-78 protein was supplied in both PBS buffer (phosphate buffered saline) filtered to yield a concentration of 2 mg/ml, and in 50 mM potassium phosphate buffer pH7.4 at 2 mg/ml. Concentrations were determined by absorbance at 280 nm. PBS buffer and potassium phosphate buffer were used as a reference for the respective samples. DSC was performed using capillary cell microcalorimeter VP-DSC (Microcal, Mass., USA), at a heating rate of 180° C./hour. A typical scan usually was from 25-90° C. for both the reference buffer and the protein sample. After each reference buffer and sample pair, the capillary cell was cleaned with a solution of 1% Decon in water followed by PBS. Resulting data traces were analysed using Origin 7 Microcal software. The DSC trace obtained from the reference buffer was subtracted from the sample trace. The precise molar concentration of the sample was entered into the data analysis routine to yield values for apparent Tm, enthalpy (ΔH) and van't Hoff enthalpy (ΔHv) values. Typically data were fitted to a non-2-state model. The DSC experiments showed that some DOM10 molecules (e.g. 10-53-474 (SEQ ID NO:2105), have higher melting temperatures compared to others (e.g. 10-275-78), see Table 9 below. Such properties are indicative of increased stability and indicate superior suitability, for example, for pulmonary delivery.

TABLE 9
Showing Apparent Tm of Both Selected dAbs DOM10-275-78 and
DOM10-53-474.

| Molecule | Apparent Tm (° C.) |
|---|---|
| DOM10-275-78 in PBS | 49.4 |
| DOM10-275-78 in | 49.8 |
| potassium phosphate | |
| DOM10-53-474 in PBS | 54.0 |

The unfolding of DOM10-53-474 protein is irreversible, and therefore the apparent Tm might be lower than the melting temperature due to some irreversible steps in the unfolding mechanism taking place before the melting point.

### Solubility:

Liquid formulations that contain high dAb concentrations are desirable for certain purposes. For example, proteins delivered therapeutically via a nebulising device may need to be at higher concentrations than would be expected for systemic delivery because not all the nebulised protein will be inhaled nor deposited in the lung. Volumes administered are also limited by the size of the reservoir in the nebuliser of interest. To this end, the solubility of both DOM10-53-474 and DOM10-275-78 was measured to determine the maximum concentration that could be achieved before incurring protein losses through aggregation and precipitation.

The proteins of a known starting concentration in PBS, determined by measuring absorbance at 280 nm, and of a known volume were each applied to a Vivaspin 20 centrifugal concentrating device, with a PES membrane of MWCO 3,000 Da (Vivasciences) and spun in a benchtop centrifuge at 4,000 g for time intervals of between 10 and 30 mins. Ten minute time periods were used initially and these were incremented as the protein became more concentrated in order to obtain the desired reduction in volume.

After each spin the protein was removed from the device, the volume measured to the nearest 50 μm using pipettes and the concentration determined. Concentration determination was performed using the absorbance reading obtained by subtracting the absorbance measured at 320 nm from the absorbance measured at 280 nm after the sample had been centrifuged at 16,000 g to remove any precipitate.

The experimental concentration was plotted against the theoretical concentration at that volume, and the maximum solubility was taken as the point at which experimental concentration diverged from theoretical.

For both proteins, a concentration of 100 mg/ml was achieved before divergence and actual protein recovery was approximately 100% of the start material.

### Downstream Processing of DOM10-53-474 and Purity Obtained:

DOM10-53-474 was expressed using a Pichia pastoris expression system and secreted in to the supernatant. The initial capture step for fermenter supernatants containing DOM10-53-474 was by direct loading onto Protein A Streamline resin (GE Healthcare) equilibrated in PBS. The resin was washed with 5-10 column volumes of PBS, followed by 2-5 column volumes 50 mM TrisHCl pH8 before eluting the protein with 4 column volumes of 0.1M Glycine pH2.0. The eluted protein was neutralised with 1M TrisHCl p118 to a final concentration of 0.2M TrisHCl. Upon neutralisation precipitation was seen, the precipitate was harvested by centrifugation and the protein resolubilised. Resolubilisation of the protein was carried out by resuspending the pellet in a volume of 10 mM TrisHCl pH7.4 equal to 1 column volume from the PrA purification step, 1M NaOH was then added to a final concentration of 75 mM to fully dissolve the precipitated protein. The pH was adjusted to pH8 by the gradual addition of 1M Glycine pH2 (final concentration ˜0.1M). Analysis by SDS-PAGE showed the resolubilised protein solution to be approximately 80% pure for DOM10-53-474. DOM10-53-474 was purified further by anion exchange chromatography. The resolubilised pellet was dialysed in to 50 mM Potassium Phosphate pH6 and loaded onto a QFF column, equilibrated with 50 mM Potassium Phosphate pH6. DOM10-53-474 was eluted using a 0-100% 50 mM Potassium Phosphate pH6+1M NaCl gradient over 20 column volumes. The fractions of the eluted peak were analysed by SDS-PAGE and the purest fractions combined and buffer exchanged into PBS. At this stage the eluted protein was approximately 97% pure as measured by SEC.

Downstream Processing of DOM10-275-78 and purity obtained:

A traditional method for initial capture and purification of antibodies and antibody fragments from fermenter supernatants or periplasmic fractions is using Protein A immobilised on an inert matrix. As an affinity chromatography step this has the advantage of good protein recovery and high (e.g. ˜90%) level of purity. However, there are some disadvantages. As with all forms of affinity chromatography some of the ligand can be leached from the column support matrix during the elution phase. Protein A is known to be a potential immunogen. Therefore, if Protein A is used, then any residual Protein A, leached from the column, should be removed or reduced as far as possible in subsequent chromatography steps.

### DOM10-275-78 Purification:

The initial capture step for either fermenter supernatants or periplasmic fraction containing DOM10-275-78 was by direct loading onto Protein A Streamline resin (GE Healthcare) equilibrated in PBS. The resin was washed with 2-5 column volumes of PBS before eluting the protein with 4 column volumes of 0.1M Glycine pH3.0. At this stage the eluted protein was approximately 99% pure, containing approximately 1% of dimeric DOM10-275-78 as measured by SEC. Protein recovery was virtually 100%. Residual PrA was measured using a PrA ELISA kit (Cygnus, #F400) and was determined to be between 50 to 200 ppm.

### Residual PrA Removal:

The residual PrA was reduced using two further chromatographic steps. The eluate from the PrA step was pH adjusted to pH6.5 using 1M Tris pH8.0 and prepared for purification on hydroxyapatite type II by addition of 1% (v/v) 0.5M sodium phosphate pH6.5 resulting in a final phosphate concentration of 5 mM. The PrA eluate was applied to the column which had been equilibrated with 5 mM phosphate pH6.5 and the DOM10-275-78 monomer eluted in the flow through. The dimer was bound to the column and eluted at the start of a salt gradient which was applied after the DOM10-275-78 had been recovered. The gradient ran from 0 to 1M NaCl in 5 mM phosphate pH6.5 over 30 column volumes. It was expected that the PrA would elute in this gradient although amounts were too small to be able to see by absorbance on the chromatogram. Complexes of PrA with the DOM10-275-78 eluted after the salt gradient when a 500 mM phosphate pH6.5 wash was applied to the column. The recovery of DOM10-275-78 monomer after this stage was measured as 74% based on absorbance at 280 nm and the purity was 100% as measured by SEC. The residual protein A levels were measured and were found to have been reduced to between 0.4 and 0.56 ppm (parts per million i.e. ng/mg).

A further purification step was introduced to reduce the residual PrA even further. The eluate pool from the hydroxyapatite column was directly applied to a phenyl (HIC) column (GE Healthcare) after addition of NaCl to a final concentration of 2M. The column had been equilibrated with 25 mM phosphate pH7.4 plus 2M NaCl. The protein was eluted with a gradient from 2M NaCl to no salt over 20 column volumes. After this step the residual PrA levels were reduced to between 0.15 to 0.19 ppm and the protein recovery was measured by absorbance at 280 nm as being 80%.

### Nebuliser Testing of DOM10-53-474 and DOM10-275-78:

### Testing of DOM10-53-474:

The nebulising device can nebulise the dAb solution into droplets only some of which will fall within the requisite size range for pulmonary deposition (1-5 μm). The particle size of the aerosol particles was analysed by laser light scattering using the Malvern Spraytek device. Two post-nebulisation samples were collected i) protein solution which remained in the reservoir and ii) aerosolized protein collected by condensation. The parameters measured to assess the nebulisation process were i) Respirable fraction—% of particle in 1-5 μm size range, this is important to determine how much dAb will reach the deep lung; ii) Particle size distribution (psd) of dAb; iii) Mean median aerodynamic diameter (MMAD)—average droplet size of nebulised dAb solution within psd. The stability of the dAb to the nebulisation process was assessed by comparing pre- and post nebulisation samples using a variety of methods, i) Size Exclusion Chromatography (SEC)—which demonstrates whether the nebulisation process caused aggregation of the dAb; ii) Sandwich EL1SA for binding to hIL-13.

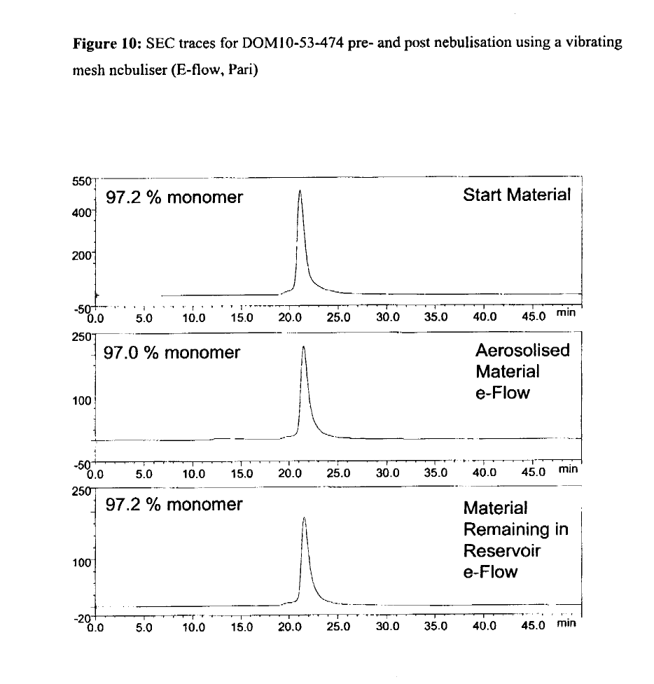

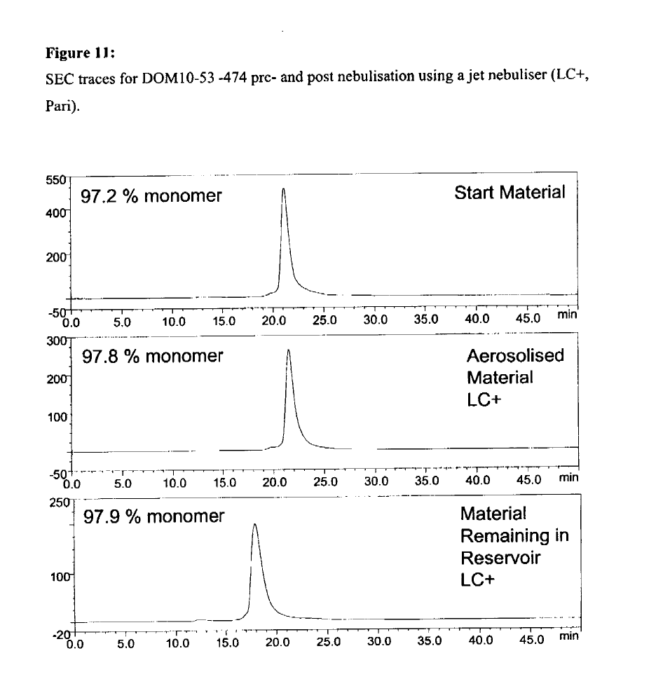

The nebulisation properties of DOM10-53-474 were investigated using both a jet nebuliser (both LC+, manufactured by Pari) and also a vibrating mesh nebuliser (both E-flow, manufactured by Pari). DOM10-53-474 protein was tested in both PBS buffer (phosphate buffered saline) at a concentration of 2.6 mg/ml, and in 25 mM sodium phosphate buffer pH7.5, 7% (v/v) PEG1000, 1.2% (w/v) sucrose at 2.3 and 4.7 mg/ml. Nebulisation was performed for approximately 3 minutes. 100 uL of protein samples (diluted to 1 mg/mL) were injected onto the SEC (TSKgel G2000SWXL, Tosoh Biosciences, Germany) column. The protein separation on SEC was performed at 0.5 mL/min for 45 minutes. PBS (phosphate buffered saline)+10% EtOH was used as mobile phase. The detection of eluting proteinaceous material was carried by on-line detection by UV (Abs 280 nm & 215 nm). The SEC profile of the pre- and two post-nebulisation samples were identical; and no peaks indicative of aggregation were seen post nebulisation see FIGS. 10 and 11. The samples were analysed for binding to hIL-13 using the Sandwich Elisa described earlier and the potency was shown to be unaffected by nebulisation, as shown in Table 10.

TABLE 10
Sandwich ELISA data for DOM10-53-474 pre- and post-
nebulisation samples. 14 - 2.3 mg/mL 25 mM sodium
phosphate buffer pH7.5, 7% (v/v) PEG1000, 1.2% (w/v)
sucrose; 15 - 4.7 mg/mL 25 mM sodium phosphate buffer
pH7.5, 7% (v/v) PEG1000, 1.2% (w/v) sucrose; 16 - 2.6 mg/mL
PBS; Cup - material remaining in cup after nebulisation; Aero -
aerosolized material. DOM10-53-474 was material in 1xPBS.

| Sample | IC₅₀ nM |
|---|---|
| DOM10-53-344 (Std) | 0.07157 |
| DOM10-53-474 | 0.04578 |
| DOM10-53-474 #14 Eflow Cup | |
| DOM10-53-474 #14 Eflow Aero | 0.03222 |
| DOM10-53-474 #14 LC+ CUP | 0.03168 |
| DOM10-53-344 (Std) | 0.12456 |
| DOM10-53-474 | 0.03556 |
| DOM10-53-474 #14 LC+ Aero | 0.03480 |
| DOM10-53-474 #15 Eflow Cup | 0.02144 |
| DOM10-53-474 #15 Eflow Aero | 0.01849 |
| DOM10-53-344 (Std) | 0.04552 |
| DOM10-53-474 | 0.02886 |
| DOM10-53-474 #15 LC+ CUP | 0.01285 |
| DOM10-53-474 #15 LC+ Aero | 0.01494 |
| DOM10-53-474 #16 Eflow Cup | 0.02242 |
| DOM10-53-344 (Std) | 0.06709 |
| DOM10-53-474 | 0.02692 |
| DOM10-53-474 #16 Eflow Aero | 0.03508 |
| DOM10-53-474 #16 LC+ Cup | 0.03386 |
| DOM10-53-474 #16 LC+ Aero | 0.02298 |

The optimum MMAD is 3 μm and for deep lung delivery the desirable respirable fraction is the highest percentage of particles <5 μm. As shown in the Table 11 below the LC+(Pari) Jet nebuliser gives the better MMAD. MMAD values are lower when the buffer contains PEG; MMAD decreases as protein concentration increases. The LC+(Pari) Jet nebuliser gives the higher %<5 μm: higher %<5 μm values are obtained when the buffer contains PEG; %<5 μm also increases as protein concentration increases.

TABLE 11
shows the MMAD and percentage of particles <5 μm
after nebulisation using LC+ (Pari) Jet nebuliser and
E-flow Rapid nebuliser:

| | eFlow Rapid | | Pari LC + | |
|---|---|---|---|---|
| | MMAD | | MMAD | % < |
| Formulation | (um) | % < 5um | (um) | 5um |
| 25 mM NaPhosphate pH7.5, | 4.26 | 60.6% | 3.98 | 61.2% |
| 7% PEG 1000, 1.2% Sucrose, | | | | |
| 2.3 mg/ml DOM10-53-474 dAb | | | | |
| 25 mM NaPhosphate pH7.5, | 4.10 | 63.8% | 3.66 | 66.5% |
| 7% PEG 1000, 1.2% Sucrose, | | | | |
| 4.7 mg/ml DOM10-53-474 dAb | | | | |
| 10-53-474, PBS, | 5.20 | 47.9% | 4.43 | 56.6% |
| 2.6 mg/ml DOM10-53-474 dAb | | | | |

### Testing of DOM10-275-78:

To determine whether this molecule possessed the requisite characteristics for effective pulmonary deposition during pre-clinical PK and efficacy studies, its properties during nebulisation were tested using a PARI LC* jet nebuliser. The properties analysed were i) the particle size distribution of the aerosol which was analysed by laser light scattering using the Malvern Spraytek; ii) Respirable fraction—% of particle in 1-5 μm size range; iii) Mass median aerodynamic diameter (MMAD)—average droplet size of nebulised dAb solution within psd. The stability of the dAb to the nebulisation process was assessed by comparing pre- and post nebulisation samples, using a variety of methods, i) Size Exclusion Chromatography (SEC)—which demonstrates whether the nebulisation process caused aggregation of the dAb; ii) Sandwich ELISA for binding to hIL-13; iii) HEK cell assay.

The dAb was tested at a concentration of 20 mg/ml in PBS as determined by u.v. absorbance at 280 nm, which represents a realistic dosing concentration. The dAb was nebulised for 30 mins with two samples collected at each of three time points during nebulisation i) protein solution which remained in the reservoir and ii) aerosolized protein collected by condensation. The three time points were 3 mins, 15 mins and 29 mins representing the beginning, middle and end of the nebulisation.

100 uL of protein samples (diluted to 1 mg/mL) were injected onto the SEC (TSKgel G2000SWXL, Tosoh Biosciences, Germany) column. The protein separation on SEC was performed at 0.5 mL/min for 45 minutes. PBS (phosphate buffered saline) was used as mobile phase. The detection of eluting proteinaceous material was carried by on-line detection by UV (Abs 280 nm & 215 nm). The SEC profiles of the nebulisation samples were identical; no peaks indicative of aggregation were seen post nebulisation. The samples were analysed for binding to hIL-13 and the potency was shown to be unaffected by nebulisation, as shown in the Table 12 below.

TABLE 12
shows potency for binding to hIL-13 of dAbs before and after
nebulisation:

| | Binding | HEK cell assay | |
|---|---|---|---|
| | Assay | EC₅₀ nM | |
| Sample | IC₅₀ nM | Cyno IL-13 | Hu IL-13 |
| DOM10-275-78 start | 1.2 | 0.93 | 0.58 |
| DOM10-275-78 cup 3 mins | 1.1 | 1.77 | 1.45 |
| DOM10-275-78 cup 15 mins | 1.2 | 1.52 | 2.18 |
| DOM10-275-78 cup 29 mins | 1.1 | 0.74 | 0.48 |
| DOM10-275-78 aerosol 3 mins | 1.1 | 0.99 | 1.8 |
| DOM10-275-78 aerosol 15 mins | 1 | 0.95 | 1.01 |
| DOM10-275-78 aerosol 29 mins | 1.1 | 0.74 | 0.62 |
| DOM10-53-474 standard | | 15.24 | 0.62 |

TABLE 13
shows Mass median aerodynamic diameter (MMAD) - average
droplet size of nebulised dAb solution. The optimum MMAD is
2-3 μm and for deep lung delivery the desirable respirable
fraction would be the highest percentage of particles <5 μm
as possible.

PARI LC*

| | MMAD | |
|---|---|---|
| Formulation | μm | % < 5 μm |
| DOM10-275-78 3 mins | 2.38 | 79.4% |
| DOM10-275-78 15 mins | 2.50 | 80.1% |
| DOM10-275-78 29 mins | 2.45 | 74.6% |

DOM10-275-78 is stable to nebulisation in the PARI LC* jet nebuliser with a high percentage of droplets <5 μm and it achieves the desired MMAD.

### Use of Nebuliser to Deliver DOM10-275-78 During Pre-Clinical PK and Efficacy Studies

### Cyno PK:

To determine the pharmacokinetic profile of DOM10-275-78 in cynomolgous monkey and thus enable a prediction of likely required clinical dosing frequency, test deliveries of DOM10-275-78 into cynomolgous monkey were made. In order to gain as comprehensive as possible understanding of the pharmacokinetics underlying this molecule when delivered to the lung, a number of different studies were performed as summarised below in Table 14:

TABLE 14
summary of study protocols

| Study | Delivery | | Number of | Method of |
|---|---|---|---|---|
| Number | Frequency | Animal Type | Animals | Delivery |
| Study 1 | Single | House dust | 6* | Sedated face |
| | | mite sensitive | | mask delivery |
| Study 2 | Single | House dust | 3 | Conscious |
| | | mite sensitive | | delivery by bell |
| | | | | device |
| Study 3 | Repeat (4 daily | House dust | 1 | Conscious |
| | doses) | mite sensitive | | delivery by bell |
| | | | | device |

*Data from only three of the study 2 animals is currently available.

Drug was delivered to sedated animals at 3 escalating doses to ensure drug tolerability (0.01, 0.1 and 1 mg/kg). Sedation was performed with 2 mg/kg Telazol in all instances. To ensure consistent levels of drug delivery, and given the differing propensity of different drug solution concentrations to nebulise the 0.01 and 0.1 mg/kg deliveries were performed over an 18 minute delivery period. The 1 mg/kg dose was delivered over 20 minutes. To compensate for anticipated higher respiratory rates and tidal volumes of the conscious bell-delivered animals, these received drug at a single dose, and only for 10 minutes. The quantity of drug nebulised was confirmed through pre- and post-delivery weighing of the filled nebuliser cup.

In order to test samples for the presence of DOM10-275-78 within the lung and serum after delivery, blood and bronchioalveolar lavage fluid (BAL) (representing a flushing of a proportion of the animals lung with phosphate-buffered saline) were collected. Blood was typically collected prior to dosing and at up to 9 timepoints within the first 24 hrs of dosing, in line with ethical guidelines. The invasive nature of BAL collection was such that it was collected only once per animal per delivery at either 1 or 24 hrs post-delivery. Blood was allowed to clot after collection before centrifugation to generate serum. Serum and BAL were then stored at −80° C. prior to analysis.

### Testing of PK Study Samples:

In order to ascertain levels of DOM10-275-78 within the samples collected, a pharmacokinetic assay was set up. A standard bind electrochemiluminescence-based plate was coated with neutravidin, blocked and then biotinylated IL-13 was added and allowed to capture to the neutravidin. Samples were then added at a range of dilutions. Any DOM10-275-78 bound was then detected using a polyclonal rabbit anti-Vh antibody followed by an anti-rabbit-sulfo-tag secondary antibody. MSD T read buffer (Mesoscale Discovery, Maryland) at 1× concentration was added and the plates were read using a sector 6000 MSD imager (Mesoscale Discovery, Maryland). Wash steps were performed between each stage of the assay.

### Results of Pharmacokinetic Study:

Serum and BAL concentrations were calculated for each timepoint collected and the resultant data for each animal was analysed using WinnonLin (Pharsight Corporation, California) pharmacokinetic analysis software. A summary of the pharmacokinetic parameters generated through this analysis are given below in Table 15:

TABLE 15
summary of the pharmacokinetic parameters

| | T½ | | | Mean |
|---|---|---|---|---|
| | (hours) | | AUC/Dose | Residence |
| Study | of dAb in | Cmax of | (0-inf), | Time (0-inf) |
| Number | animal | dAb (ng/ml) | (hr * kg * ng/mL/mg) | (hrs) |
| Study 1 | 4.5 | 173.9 | 1689.5 | 6.7 |
| Study 2 | 4.1 | 249.5 | 1529.1 | 5.8 |
| Study 3 | 3.7 | 347.9 | 1987.9 | 5.5 |

Mean serum concentrations for each study at a number of timepoints are summarised below in Table 16:

TABLE 16
Mean serum concentrations for each study 1-3 at
a number of timepoints

| | | | | Concentration |
|---|---|---|---|---|
| | | Concentration | | at 24 hours |
| | | at 2 hours post- | Concentration at 8 | post- |
| Study | Cmax | delivery | hours post- | delivery |
| Number | (ng/ml) | (ng/ml) | delivery (ng/ml) | (ng/ml) |
| Study 1 | 173.9 | 144.5 | 68.2 | 6.8 |
| Study 2 | 249.5 | 217.6 | 65.5 | 6.2 |
| Study 3 | 347.9 | 347.9 | 127.3 | 5.9 |

In addition analyses of the levels of drug present in the BAL after 24 hrs was determined and adjusted for dilution using urea level determination to give a figure expressed per ml of epithelial lining fluid. These results are given below in Table 17:

TABLE 17
Levels of DOM10-275-78 in bronchioalveolar
lavage fluid (BAL)

| Study | 24 hour timepoint (mg |
|---|---|
| Number | DOM10-275-78/ml ELF) |
| Study 1 | 1.23 |
| Study 2 | 4.52 |
| Study 3 | 4.75 |

In conclusion significant levels of domain antibody can be delivered to the cynomolgous monkey lung and these remain considerable after 24 hrs. In addition they show little, if any accumulation after repeat administration on a daily basis. Allergic animals typically had maximal serum drug concentrations of around200 ng/millilitre and these were highest in animals to whom drug was delivered when conscious. Highest levels were typically achieved 2-4 hrs post-delivery. The half-life of DOM10-275-78 was around 4 hours in all cases, and significantly therapeutically relevant levels of drug were present in both lung and serum 24 hours of delivery, supporting potential daily administration.

Nebuliser delivery of Alternative dAb Formats:

The hydrodynamic size of a ligand (e.g., dAb monomer) and its serum half-life can also be increased by conjugating or linking the ligand to a binding domain (e.g., antibody or antibody fragment) that binds an antigen orepitope that increases half-life in vivo. For example, the ligand (e.g., dAb monomer) can be conjugated or linked to an anti-serum albumin dAb or to the Fc domain of a human IgG molecule. Also other biologically active peptides e.g. exendin-4, can improve their in-vivo half-life by conjugation with an anti-serum albumin dAb.

PK studies have demonstrated that dAbs delivered to the lungs are readily and rapidly transferred into the circulation. Therefore pulmonary delivery offers the potential for an easy and efficacious route of administration for alternative dAb formats with longer serum half-lives, if they can demonstrate the requisite characteristics.

Therefore two alternative dAb formats were tested in two different nebuliser devices to determine whether alternative dAb formats could be nebulised successfully. The dAbs tested were DMS1529, an anti-VEGF dAb conjugated with human Fc; and DAT0115, an anti-human serum dAb conjugated to exendin-4 peptide and this binds to the GLP receptor and to human albumin.

### Production of DMS1529:

DMS1529 was expressed in CHO cells in serum/protein free media at a titre of ˜0.5 mg/ml. The culture supernatant was 0.2 μm filtered and purified by affinity chromatography as follows. An XK50 column was packed to 20 cm bed height with Mab Select Sure resin (GE Healthcare) and equilibrated into 1×PBS at 30 ml/min. The column was washed with 2CV (0.8 L) of 0.1M NaOH and re-equilibrated with 4CV (1.6 L) 1×PBS.

10 L of DMS1529 supernatant was then applied at 30 ml/min (cm/h). Post the supernatant load the column was washed with 4CV (1.6 L) of 1×PBS, 2CV (0.8 L) 1×PBS supplemented with 0.85M NaCl and 4CV (1.6 L) of 10 mM Sodium Acetate, pH5.5. The protein was eluted using 0.1M Sodium Acetate, pH3.6. Collection started as the OD280 reached 20 mAus and was halted as it fell below 35 mAus. The elution peak was 600 ml with a pH of 3.7. It was held at this pH for 1 hour prior to neutralization to pH4.5 with 2M Sodium Acetate. The solution turned cloudy as the pH approached 4.5. The protein was then filtered through a SartoBran-P depth filter and the OD measured. The protein pool was then filter sterilized and stored at 4 degrees.

The final protein pool was at a concentration of 6 mg/ml and the purity was measured as being 96.4% by SEC. 100 μl of protein sample was loaded onto a TSK3000 SWXL SEC column (Tosoh, Germany) which was run at 0.5 ml/min for 30 mins in 0.1M Na phosphate/400 mM NaCl pH6.8 mobile phase. 40 mls of this protein was buffer exchanged into a more stable formulation comprising 10 mM sodium acetate/2% glycine/0.05 mM EDTA/0.04% Tween 80/10% sucrose pH5, using three 53 ml CV desalting columns in series (GE Healthcare) and running at 5 ml/min. The buffer exchanged protein was concentrated using a Vivaspin centrifugal concentrator with MWCO 3,000 Da (Vivasciences) to 9.8 mg/ml and stored at 4° C. before being tested in the nebulisers.

### Production of DAT0115:

1 L of frozen E. coli culture supernatant produced in a 5 L fermenter was defrosted at room temperature and at 4 degrees overnight. The Akta Xpress Module (a purification system obtained from GE Healthcare, UK) was sanitised by standing overnight in 1M NaOH and the following morning the Akta was washed into sterile, endo free dulbeccos PBS. The defrosted supernatent was centrifuged at 16,000 rpm and then 0.2 μm filtered. A 200 ml Protein L streamline column was cleaned with 200 ml of 6M Guanidine HCl and then washed into PBS. Defrosted supernatant was applied to the column at 20 ml/min, the column was then washed with dulbeccos PBS until the A280 was below 15 mAus and then washed with approx. 400 ml of 20 mM Tris, pH 7.4. The column was eluted using 0.1M Glycine, pH2 and the elution peak was collected in two fractions, the main peak comprising the peak maximum and some of the trailing tail (200 ml) and the elution tail comprising the rest of the elution tail (200 ml). Both elution fractions were neutralised by addition of ⅕^{th} final volume of 1M Tris, pH7.4, the pH was checked with a pH strip and the fractions were filter sterilised and stored at four degrees.

The fraction containing the peak maximum was concentrated to 8.3 mg/ml and used for nebuliser testing.

### Nebulisation of Alternative dAb Formats:

The different dAb formats were tested at ˜10 mg/ml for a period of up to 30 mins. A Malvern Spraytek was used to investigate the particle size distribution, MMAD and percentage of particles (droplets)<5 μm to determine their pulmonary deposition characteristics.

Both formatted dAbs were tested using a jet nebuliser (LC+, Pari) and a vibrating mesh jet nebuliser (E-flow, Pari). During the testing samples were taken from both the nebuliser sample reservoir (cup) and from collected condensate of aerosolised dAb at three time points and assessed for stability using i) SEC-HPLC to determine any increase in aggregation; and ii) potency assay to assess any decrease in activity. The time points sampled were 3 min, 15 mins and 29 mins for samples nebulised with the LC+nebuliser and only 3 mins for the e-Flow nebuliser as this device nebulises samples much more rapidly as shown in the table 18 below.

TABLE 18
Nebulisation rate of formatted dAbs

| | E-Flow | LC+ |
|---|---|---|
| Nebulised Sample | (ml/min) | (ml/min) |
| DMS1529 10 mg/ml Start (3 minutes) | | 0.17 |
| DMS1529 10 mg/ml Middle (15 minutes) | | 0.18 |
| DMS1529 10 mg/ml End (27 minutes) | | 0.15 |
| DMS1529 10 mg/ml (3 minutes) | 0.76 | |
| DAT0115 8.3 mg/ml Start (3 minutes) | | 0.31 |
| DAT0115 8.3 mg/ml Middle (15 minutes) | | 0.23 |
| DAT0115 8.3 mg/ml End (27 minutes) | | 0.13 |
| DAT0115 8.3 mg/ml (3 minutes) | 0.47 | |

The results of the study show that DMS1529 is nebulised at a fairly constant rate throughout the 30 mins nebulisation time whereas the nebulisation rate for DAT0115 appears to decrease with time, as the volume is reduced. Therefore the nebulisation rate for the desired clinical delivery time would have to be measured for this molecule.

To determine the protein stability and propensity to aggregate during nebulisation 100 μL of protein samples were injected onto the SEC column. For DMS1529, the samples were diluted to 1 mg/ml with 0.1M Na phosphate/400 mM NaCl pH6.8 and the column used was TSKgel G3000SWXL, (Tosoh Biosciences, Germany). The protein separation was performed at 0.5 mL/min for 45 minutes. 0.1M Na phosphate/400 mM NaCl pH6.8 was used as mobile phase. For DAT0115 the column used was a Superdex 200 10/300 column (GE Healthcare). The protein separation was performed at 0.5 mL/min for 50 minutes. 100 μl of neat sample at ˜8.3 mg/ml in 200 mM Tris/80 mM Glycine pH 7.4 were injected and 20 mM Sodium Citrate pH6.2, 100 mM NaCl was used as mobile phase. The detection of eluting proteinaceous material from either column was by on-line detection by UV (Abs 280 nm & 215 nm). The SEC profiles of the pre- and post-nebulisation samples were essentially identical; and no peaks indicative of any increased aggregation were seen post nebulisation. The percentage of monomer in each sample was calculated from the integrated chromatograms. The samples were analysed for binding in their respective activity assays and the potency was shown to be unaffected by nebulisation, as shown in Table 19.

TABLE 19
Binding assay data for DMS1529 Fc pre- and post-
nebulisation samples and cell assay data for DAT0115.

| Sample | |
|---|---|
| | EC₅₀ (nM) |
| DMS1529 (std) | 0.088 |
| DMS1529Fc T0 | 0.138 |
| DMS1529Fc e-Flow cup 3 mins | 0.15 |
| DMS1529Fc e-Flow aerosol 3 mins | 0.164 |
| DMS1529Fc LC+ cup 3 mins | 0.137 |
| DMS1529Fc LC+ cup 15 mins | 0.12 |
| DMS1529Fc LC+ cup 29 mins | 0.128 |
| DMS1529Fc LC+ aerosol 3 mins | 0.134 |
| DMS1529Fc LC+ aerosol 15 mins | 0.12 |
| DMS1529Fc LC+ aerosol 29 mins | 0.095 |
| | EC₅₀ (pM) |
| DAT0115 (std) (LH200208) | 310.5 |
| DAT0115 T0 | 178.2 |
| DAT0115 e-Flow cup 3 mins | 535.1 |
| DAT0115 e-Flow aerosol 3 mins | 797.8 |
| DAT0115 LC+ cup 3 mins | 725.1 |
| DAT0115 LC+ cup 15 mins | 453.3 |
| DAT0115 LC+ cup 29 mins | 445.4 |
| DAT0115 LC+ aerosol 3 mins | 295.6 |
| DAT0115 LC+ aerosol 15 mins | 316.4 |
| DAT0115 LC+ aerosol 29 mins | 141.8 |

The potency assay used for DMS1529 was DOM15 Fc binding assay and that for DAT0115 was DAT01 cell assay. These assays are described below.

The optimum MMAD is ˜3 μm and for deep lung delivery the desirable respirable fraction would be the highest percentage of particles <5 μm as possible. These measurements for both dAb formats in both nebulisers are shown in table 20.

TABLE 20
Measurements of MMAD and percentage of
particles <5 μm for formatted dAbs:

| | MMAD | |
|---|---|---|
| Sample | (μm) | % < 5 μm |
| DMS1529Fc e-Flow 3 mins | 2.98 | 82.9% |
| DMS1529Fc LC+ 3 mins | 1.25 | 70.2% |
| DMS1529Fc LC+ 15 mins | 1.84 | 65.9% |
| DMS1529Fc LC+ 29 mins | 1.25 | 76.6% |
| DAT0115 e-Flow 3 mins | 2.93 | 85.0% |
| DAT0115 LC+ 3 mins | 2.55 | 56.1% |
| DAT0115 LC+ 15 mins | 1.22 | 63.6% |
| DAT0115 LC+ 29 mins | 2.11 | 56.4% |

Both molecules are nebulised effectively in both nebulisers. For both molecules the e-Flow nebuliser produces a greater number of droplets which are <5 μm although the MMAD is higher for the e-Flow than for the LC+. This would indicate a tighter PSD with the e-Flow than observed with the LC+. Both molecules are nebulised with a consistent MMAD and %<5 μm for up to 30 minutes in the LC+. For DAT0115 the difference between the two methods of nebulisation, jet or vibrating mesh, is more significant than for DMS1529. DAT0115 nebulises more effectively in the e-Flow vibrating mesh nebuliser with an MMAD of ˜3 μm and 85% of droplets in the respirable fraction.

### DMS1529 Binding Assay:

Recombinant human VEGF is captured onto Nunc maxisorp ELISA plates and then non specific binding blocked with 1% BSA in PBS. The plates are then incubated with the dms 1529 (dAb-hFc) for one hour. Binding is detected using a peroxidase anti-human IgG (Fc specific) antibody. After one hour the plates are developed using SureBlue TMB substrate (KPL) and absorbance read at 450 nm.

### DAT01 Cell Assay:

This assay uses CHO cells stably transfected with the 6CRE/luciferase reporter gene (CHO-luc) (GSK). On production of cAMP following GLP-1 activation of the receptor, the promoter gene (containing 6 copies of cAMP response element—6CRE) drives the expression of the luciferase reporter gene. This then catalyses a reaction with luciferin to produce light which can be measured on a plate reader. Briefly, CHO-luc cells are left to adhere to tissue culture plates then dAb sample is added. After 3 hours Bright Glo luciferase reagent (Promega) is added to the wells and luminescence measured on a plate reader.

Nebulisation of DOM 1 h-131-206:

These experiments were carried out to determine whether the domain antibody DOM 1h-131-206 may be nebulised for oral inhalation. Since the suitability of the nebulised dose for oral inhalation is highly dependant certain key physical parameters, experiments were also carried out to investigate the effects of nebulisation of DOM 1h-131-206 in terms of droplet size distribution, concentration of the active and the active components stability (fragmentation/aggregation).

### Method:

### To Determine Effect of Nebulising Formulation

The Pari eFlow was used to nebulise a solution comprising 9.7 mg/ml DOM1 h-131-206 in 20 mM Acetate Buffer p15.5 with 4% Lutrol L44, 0.5% Arginine, 0.01% Polysorbate 80 and 0.682% NaCl.

The aerosol output from the nebuliser was collected in a twin impinger apparatus, set to pull a flow rate of 60 L/min when connected to an auxiliary pump. (European Pharmacopoeia section 2.9.18). The set up for the experiment was as follows. The nebuliser mouthpiece was placed directly in front and inline with the inlet of the twin impinger throat located 4 cm away. The twin impinger had 7 ml of buffer in stage 1 and 30 ml of the same in stage 2; the composition of the buffer used in this experiment was 20 mM Acetate Buffer at pH5.5 with 0.5% Arginine and 0.682% NaCl.

In sequence, the Pari eFlow was filled with 6 ml of the drug solution. The pump connected to the twin impinger apparatus was turned on followed by the nebuliser, whereupon the dose from the nebuliser entered and was collected by the twin impinger apparatus. To collect multiple samples, the nebuliser was run for 2 minutes, after which the nebuliser was turned off and the twin impinger apparatus was replaced. The nebuliser was turned on to collect for a further 2 minutes from the same material in the nebuliser. Samples from each of the twin impinger apparatus was quantitatively washed down into 100 ml volumetric flasks for stages 1 and 2 respectively. The wash down was performed using buffer (as described above). The samples were run using Size Exclusion Chromatography (SEC) to determine the concentration of the monomer peak, and to observe if the sample had undergone fragmentation/aggregation upon dosing. Determination of concentration of the monomer peak was possible by comparison with analytical standard solutions of known concentration.

TABLE 21
shows the method details for the SEC:

| | |
|---|---|
| Mobile phase | 100 mM Sodium Phosphate, 400 mM NaCl, |
| | pH6.8, with 10% n-propanol |
| Flow rate | 0.5 ml/min |
| Column temperature | not controlled |
| UV detection wavelength | 280 nm |
| Injection volume | 10 μl |
| Analysis time | 40 minutes |

### To Determine Proplet Size Distribution (DSD) of the Nebulised Plume:

The equipment used to measure DSD was a Malvern Spraytec (Malvern Instruments UK, model STP5311), a non-invasive system which measures droplet size by laser light scattering.

The nebuliser mouthpiece was placed 1 cm away from the laser beam and 3 cm away from the receiving optical lens. A twin impinger apparatus was set at 70 L/min to direct the nebulised plume through the laser's measurement zone. The mouthpiece of the twin impinger apparatus was placed 1 cm from the laser beam, in the direction of travel of the nebulised plume.

The plume of the Pari eFlow is dense, causing problems with beam steering. Beam steering is observed when the density of air (and hence its reflective index) in the measurement zone becomes significantly different to ambient air, such as in the presence of a significant concentration of propellant gas or another vapour. When this occurs, the laser bean is deflected from its alignment in ambient conditions, resulting in an erroneous reading for large particles. Therefore the detectors affected by beam steering were disabled. This does reduce the size range of the analysis, as large particles will not be able to be detected. These particles are however not present in these samples.

The evaporation of water from the nebulised drops in the measurement zone causes ‘beam steering’ where some very large droplets were perceived to be present. Detectors were turned off until the Bimodal distribution disappeared, this meant detectors 1-12 of the light scattering apparatus were disabled. The measurements were run using the continuous measurement technique with a measurement being taken every 5 seconds.

In sequence, the Pari eFlow was filled with 6 ml of the drug solution. The pump connected to the twin impinger apparatus was turned on followed by initialisation of the measurement. The nebuliser was then turned on, whereupon the dose from the nebuliser passed through the measurement zone of the Spraytec and was collected by the twin impinger apparatus. To collect multiple samples, the nebuliser was run for 2 minutes, after which the nebuliser was turned off, the measurement was stopped and the twin impinger apparatus was replaced. The nebuliser was turned on to collect for a further 2 minutes from the same material in the nebuliser.

### Results

TABLE 22
Mass of active in Stages 1 and 2 of the twin impinger
determined by SEC vs. nebulisation time

| | |
|---|---|
| | Mass of DOM |
| Twin Impinger stage | 1h-131-206 (mg) |

| (n = 1) | 0-2 mins | 2-4 mins |
|---|---|---|
| 1 | 4.18 | 4.33 |
| 2 | 10.79 | 10.49 |
| Total | 14.97 | 14.82 |

The results in table 22 above show that a consistent mass has been delivered over time, despite some inherent variability in output rate of the nebuliser.

TABLE 23
Concentration of active (monomer) as
a % area determined by SEC

| | Mass of DOM |
|---|---|
| | 1h-131-206 (% area) |
| | Input material |
| Twin Impinger stage | 96.93 |

| (n = 1) | 0-2 mins | 2-4 mins |
|---|---|---|
| 1 | 95.39 | 95.84 |
| 2 | 96.76 | 96.69 |

The results in table 23 show that the nebulised samples have a comparable amount of DOM1 h-131-206 to the input material. This suggests that the material does not undergo significant fragmentation or aggregation on nebulisation when this data is used in conjunction with the quantitative concentration data.

TABLE 24
Droplet size distribution of nebulised active

Timepoint

| | 0-2 mins | 2-4 mins |
|---|---|---|
| Dv(10) | 1.377 | 1.713 |
| Dv(50) | 3.617 | 3.860 |
| Dv(90) | 7.573 | 7.819 |
| % < 10 μm | 97.045 | 96.615 |
| % < 5 μm | 70.029 | 67.081 |
| % < 2 μm | 19.717 | 14.675 |

The droplet size distribution above in table 24 shows some slight coarsening in the second timepoint, but this is a common trait in nebulised formulations. The maximum change in the Dv values is 0.34 μm which is unlikely to have an impact on the in vivo dose.

In conclusion, the results presented show that the active dAb can successfully be nebulised using the Pari eFlow nebuliser with little effect on the concentration and composition of the formulation.

### Conclusion:

It has been demonstrated as described above that polypeptides such as dAbs can be nebulised in a range of commercially available nebuliser devices and importantly that they retain stability and biological activity after nebulisation and there is no significant aggregation observed following nebulisation. When viscosity enhancing excipients, such as PEG are added to the buffer formulation, particle size distribution and percentage droplet size less than 5 μm can be improved, thus potentially improving dAb delivery to the deep lung.

Delivery of dAb to the lung can also be further improved by increasing the dAb concentration for example a concentration of up to about 40 mg/ml and delivery time without any reduction in dAb stability or activity.

1. A composition which comprises or consists of (a) a polypeptide and (b) a physiologically acceptable buffer, and wherein the composition comprises liquid droplets and about 40% or more of the liquid droplets present in the composition have a size which is less than about 6 microns.

2. A composition according to claim 1, wherein about 40% or more of the liquid droplets present in the composition have a size in the range from about 1 to about 6 microns.

3. A composition according to claim 1, wherein about 40% or more of the liquid droplets present in the composition have a size which is less than about 5 microns.

4. A composition according to claim 1, wherein the polypeptide comprises or consists of a polypeptide domain.

5. A composition according to claim 1, wherein the polypeptide comprises or consists of an immunoglobulin molecule.

6. A composition according to claim 1, wherein the polypeptide comprises or consists of up to 150 amino acids.

7. A composition according to claim 1, wherein the polypeptide comprises or consists of a domain antibody (dAb).

8. A composition according to claim 1, wherein the polypeptide comprises or consists of a non-Ig scaffold.

9. A composition according to claim 1, wherein the polypeptide has a melting temperature (TM) in the range from about 55 deg C. to about 90 deg C.

10. A composition according to claim 1, wherein the buffer has a pH range of between about 4 and about 8.

11. A composition according to claim 1, wherein the buffer has a viscosity which is about equal to the viscosity of a solution of about 2% to about 10% PEG 1000 in 50 mM phosphate buffer containing 1.2% (w/v) sucrose.

12. A composition which comprises or consists of (a) a polypeptide and (b) a physiologically acceptable buffer, and wherein the buffer has a pH range of between about 4 and about 8, and a viscosity which is about equal to the viscosity of a solution of about 2% to about 10% PEG 1000 in 50 mM phosphate buffer containing 1.2% (w/v) sucrose.

13. A composition according to claim 12, wherein the polypeptide comprises or consists of a polypeptide domain e.g. wherein the polypeptide domain is present as a monomer.

14. A composition according to claim 12, wherein the polypeptide comprises or consists of an immunoglobulin molecule.

15. A composition according to claim 12, wherein the polypeptide comprises or consists of up to 150 amino acids.

16. A composition according to claim 12, wherein the polypeptide comprises or consists of a domain antibody (dAb).

17. A composition according to claim 12, wherein the polypeptide comprises or consists of a non-Ig scaffold.

18. Composition according to claim 12, wherein the polypeptide has a melting temperature (TM) in the range from about 55 deg C. to about 90 deg C.

19. A composition according to claim 12, wherein about 40% or more of the liquid droplets present in the composition have a size less than about 6 microns.

20. A composition according to claim 19, wherein about 40% or more of the liquid droplets present in the composition have a size in the range from about 1 to about 6 microns.

21. A composition according to claim 19, wherein about 40% or more of the liquid droplets present in the composition have a size which is less than about 5 microns.

22. A composition according to claim 1, wherein the buffer is a phosphate, acetate, citrate or histidine buffer and optionally further comprises (a) additional agents to increase viscosity and/or (b) stabilising agents.

23. A composition according to claim 1, wherein the polypeptide can bind to a target molecule in pulmonary tissue.

24. A composition according to claim 23, wherein the target is selected from a TNF receptor, TNFR1, IL-1, IL-1R, IL-4, IL-4R, IL-5, IL-6, IL-6R, IL-8, IL-8R, IL-9, IL-9R, IL-10, IL-12 IL-12R, IL-13, IL-13Ra2, IL-15, IL-15R, IL-16, IL-17R, IL-17, IL-18, IL-18R, IL-23 IL-23R, IL-25, CD2, CD4, CD11a, CD23, CD25, CD27, CD28, CD30, CD40, CD40 L, CD56, CD138, ALK5, EGFR, FcER1, TGFb, CCL2, CCL18, CEA, CR8, CTGF, CXCL12 (SDF-1), chymase, FGF, Furin, Endothelin-1, Eotaxins (e.g., Eotaxin, Eotaxin-2, Eotaxin-3), GM-CSF, ICAM-1, ICOS, IgE, IFNa, 1-309, integrins, L-selectin, MIF, MIP4, MDC, MCP-1, MMPs, neutrophil elastase, osteopontin, OX-40, PARC, PD-1, RANTES, SCF, SDF-1, siglec8, TARC, TGFb, Thrombin, Tim-1, TNF, TNFR1, TRANCE, Tryptase, VEGF, VLA-4, VCAM, α4β7, CCR2, CCR3, CCR4, CCR5, CCR7, CCR8, alphavbeta6, alphavbeta 8, cMET, and CD8.

25. A composition according to claim 24, wherein the polypeptide is an anti-TNF receptor domain antibody (dAb).

26. A composition according to claim 25, wherein the anti-TNF receptor dAb is selected from Dom 1 h-131, Dom 1 h-131-8, Dom 1 h-131-24, Dom 1 h-131-53, Dom 1 h-131-70, Dom 1 h-131-83, Dom 1 h-131-117, Dom 1 h-131-151, Dom 1 h-131-511, Dom 1 h-131-202, Dom 1 h-131-206, Dom 1 h-131-201, 1 h-131-203, 1 h-131-204 and 1 h-131-205.

27. A composition according to claim 26, wherein the anti-TNF receptor dAb comprises an amino acid sequence that is at least 93% identical to the amino acid sequence of DOM1h-131-206 (shown in FIG. 1).

28. A composition according to claim 26, wherein the anti-TNF receptor dAb comprises an amino acid sequence that is at least 95% identical to the amino acid sequence of DOM1h-131-511 (shown in FIG. 1).

29. A composition according to claim 24, wherein the dAb binds IL-13 and comprises an amino acid sequence which is identical to an amino acid sequence disclosed in FIG. 12b (Dom 10-53-474) or FIG. 12c (Dom 10-275-78) or which comprises a sequence which has 80% identity with an amino acid sequence disclosed in FIG. 12b or FIG. 12c, 85%, 86%, 87%, 88%, 89%, 90%, 91%, 92%, 93%, 94% 95%, 96% or 97% identity.

30. A composition according to claim 24, wherein the dAb binds IL-1R1 and comprises an amino acid sequence which is identical to an amino acid sequence disclosed in FIG. 12d (Dom 4-130-202) or FIG. 12e (Dom 4-130-201) or which comprises a sequence which has 80% identity with an amino acid sequence disclosed in FIG. 12d or FIG. 12e, 85%, 86%, 87%, 88%, 89%, 90%, 91%, 92%, 93%, 94% 95%, 96% or 97% identity.

31. A composition according to claim 30, wherein the dAb comprises an amino acid sequence that is at least 97% identical to the amino acid sequence of Dom 4-130-202 (shown in FIG. 12d).

32. A composition according to claim 30, wherein the dAb comprises an amino acid that is at least 98% identical to the amino acid sequence of Dom 4-130-202 (shown in FIG. 12e).

33. A composition according to claim 24, wherein the dAb binds VEGF and comprises an amino acid sequence which is identical to an amino acid sequence disclosed in FIG. 14 or which comprises a sequence which has e.g. 80% identity with an amino acid sequence disclosed in FIG. 14, 85%, 86%, 87%, 88%, 89%, 90%, 91%, 92%, 93%, 94% 95%, 96% 97%, 98% or 99% identity.

34. A composition according to claim 33, wherein the dAb comprises an amino acid sequence that is at least 97% identical to the amino acid sequence of DOM15-26-593 (shown in FIG. 14a).

35. A composition according to claim 33, wherein the dAb comprises an amino acid that is at least 97% identical to the amino acid sequence of DOM15-26-593 (shown in FIG. 14a), and which further comprises a domain of an antibody constant region.

36. A composition according to claim 24, wherein the dAb binds IL-13 and comprises an amino acid sequence which is identical to an amino acid sequence disclosed in FIG. 12b (Dom 10-53-474) or FIG. 12c (Dom 10-275-78) or which comprises a sequence which has 80% identity with an amino acid sequence disclosed in FIG. 12b or FIG. 12c, 85%, 86%, 87%, 88%, 89%, 90%, 91%, 92%, 93%, 94% 95%, 96% or 97% identity.

37. A composition according to claim 23, wherein the polypeptide can bind to a systemic target molecule, e.g. wherein the systemic target molecule is at least one selected from the group consisting of: a systemic target molecule selected from human or animal proteins, cytokines, cytokine receptors, enzymes co-factors for enzymes or DNA binding proteins such as ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1α, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, f3-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, exendin and GLP-1.

38. A composition according to claim 13 wherein the dAb can bind to human albumin and it is linked to an exendin or GLP molecule.

39. A composition according to claim 13, wherein polypeptide molecule comprises the Exendin 4 (G4S)₃ DOM7h-14 fusion (DAT0115) or any molecule which has 80% identity with dat0115 amino acid sequence 85%, 90%, 91%, 92%, 93%, 94% 95%, 96%, 97%, 98%, or 99% identity.

40. A composition according to claim 1 which further comprises a pharmaceutically acceptable carrier, diluent, or excipient.

41. A composition according to claim 1, for use in medicine.

42. A composition according to claim 1, for delivery of a polypeptide to the lung or e.g. for systemic delivery of a polypeptide.

43. A composition according to claim 1, for the treatment, prevention or diagnosis of a lung or respiratory condition or disease.

44. A composition according to claim 1 wherein the dAb is a formatted dAb.

45. A method of treating, preventing or diagnosing a lung or respiratory condition or disease comprising the step of contacting a subject with composition according to claim 1.

46. A method of treating, preventing or diagnosing a deep lung condition or disease comprising the step of contact a subject with a composition according to claim 12.

47. A method of delivering a polypeptide to the deep lung tissues of a subject comprising the step of contacting a subject with a composition according to claim 12.

48. A method of delivering a desired molecule to a subject, which comprises administering a composition according to claim 1 directly to the lungs of a subject.

49. A method for treating, preventing or diagnosing a lung condition or respiratory disorder, which comprises administering a composition according to claim 1 directly to the pulmonary tissue of a subject.

50. A method according to claim 35, wherein the composition is administered at a daily dosage of between about 5 mg per Kg to about 0.005 mg per Kg body weight of a subject to be treated.

51. A method according to claim 48, wherein the composition is administered to a subject using a nebuliser, inhaler or intranasal device.

52. A nebuliser, inhaler or intranasal device, which comprises the composition of claim 1.

53. A method of delivering a composition of claim 1 to pulmonary tissue in a subject using a nebuliser, inhaler or intranasal device.

54. A process for producing a pharmaceutical composition, e.g. for treating, preventing or diagnosing a lung condition or disease, which comprises mixing (a) a composition according to claim 1, with (b) a pharmaceutically acceptable carrier, diluent or excipient.

55. A process for producing a polypeptide composition for treating, preventing or diagnosing a lung condition or disease which comprises mixing (a) a polypeptide with (b) a physiologically acceptable buffer which has a pH range of between about 4 and about 8 and a viscosity which is about equal to the viscosity of a solution of about 2% to about 10% PEG 1000 in 50 mM phosphate buffer containing 1.2% (w/v) sucrose.

56. A process according to claim 55, wherein the polypeptide comprises or consists of a domain antibody (dAb).

57. A process for producing the composition of claim 1 which comprises the steps of (a) mixing a polypeptide with a physiologically acceptable buffer, and then (b) passing the polypeptide and buffer composition from step (a) through a nebuliser, inhaler or intranasal delivery device.

58. A process according to claim 57, wherein the physiologically acceptable buffer has a pH range of between about 4 and about 8 and a viscosity which is about equal to the viscosity of a solution of about 2% to about 10% PEG 1000 in 50 mM phosphate buffer containing 1.2% (w/v) sucrose.

59. A method of manufacturing a polypeptide composition for pulmonary delivery comprising the step of using a physiologically acceptable buffer which has a pH range of between about 4 and about 8 and a viscosity which is about equal to the viscosity of a solution of about 2% to about 10% PEG 1000 in 50 mM phosphate buffer containing 1.2% (w/v) sucrose.

60. The method of claim 47, wherein the polypeptide composition comprises or consists of a domain antibody (dAb).


SEQUENCE LISTING (27 sequences)

SEQ ID NO: 1 - protein, 119 aa, Homo sapiens
EVQLLESGGGLVQPGGSLRLSCAASGFTFAHEPMVWVRQAPGKGLEWVSHIDRVGQDTYYADSVKGRFTISRDNSKNTLYLQMNSLRAEDTAVYYCAKLPKRGPRFDYWGQGTLVTVSS

SEQ ID NO: 2 - protein, 119 aa, Homo sapiens
EVQLLESGGGLIQPGGSLRLSCAASGFTFAHETMVWVRQAPGKGLEWVSHIDRVGQDTYYADSVKGRFTISRDNSKNTLYLQMNSLRAEDTAVYYCAKLPKRGPWFDYRGQGTLVTVSS

SEQ ID NO: 3 - protein, 119 aa, Homo sapiens
EVQLLESGGGLVQPGGSLRLSCAASGFTFAHETMVWVRQAPGKGLEWVSHIDRVGQDTYYADSVKGRFTISRDNSKNTLYLQMNSLRAEDTAVYYCAKLPKRGPWFDYRGQGTLVTVSS

SEQ ID NO: 4 - protein, 119 aa, Homo sapiens
EVQLLESGGGLIQPGGSLRLSCAASGFTFAHETMVWVRQAPGKGLEWVSHIDRVGQDPYYADSVKGRFTISRDNSKNTLYLQMNSLRAEDTAVYYCALLPKRGPWFDYRGQGTLVTVSS

SEQ ID NO: 5 - protein, 119 aa, Homo sapiens
EVQLLESGGGLVQPGGSLRLSCAASGFTFAHETMVWVRQAPGKGLEWVSHIDRVGQDTYYADSVKGRFTISRDNSKNTLYLQMNSLRAEDTAVYYCALLPKRGPWFDYWGQGTLVTVSS

SEQ ID NO: 6 - protein, 119 aa, Homo sapiens
EVQLLESGGGLVQPGGSLRLSCAASGFTFAHETMVWVRQAPGKGLEWVSHIDRVGQDPYYADSVEGRFTISRDNSKNTLYLQMNSLRAEDTAVYYCAVLPKRGPWFDYWGQGTLVTVSS

SEQ ID NO: 7 - protein, 119 aa, Homo sapiens
EVQLLESGGGLIQPGGSLRLSCAASGFTFAHETMVWVRQAPGKGLEWVSHIDGGGVDTYYADPVKGRFTISRDNSKNTLYLQMNSLRAEDTAVYYCALLPKRGPWFDYRGQGTLVTVSS

SEQ ID NO: 8 - protein, 119 aa, Homo sapiens
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYYCALLPKRGPWFDYWGQGTLVTVSS

SEQ ID NO: 9 - protein, 119 aa, Homo sapiens
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSS

SEQ ID NO: 10 - protein, 163 aa, Artificial
  Fusion
HGEGTFTSDLSKQMEEEAVRLFIEWLKNGGPSSGAPPPSGGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 11 - protein, 118 aa, Homo sapiens
GVQLLESGGGLVQPGGSLRLSCAASGFTFAWYDMGWVRQAPGKGLEWVSSIDWHGEVTYYADSVKGRFTISRDNSKNTLYLQMNSLRAEDTAVYYCATAEDEPGYDYWGQGTLVTVSS

SEQ ID NO: 12 - protein, 116 aa, Homo sapiens
EVQLLESGGGLVQPGGSLRLSCVASGFTFDVAEMDWVRQAPGKGLEWVSTISPSRRGTYYADSVKGRFTISRDNSKNTLYLQMNSLRAEDTAVYYCAKAYTGRSLWGPGTLVTVSS

SEQ ID NO: 13 - protein, 108 aa, Homo sapiens
DIQMTQSPSSLSASVGDRVTISCRASQDIYLNLDWYQQKPGKAPKLLISFGSELQSGVPSRFSGSGYGTDFTLTISSLQPEDSATYYCQPSFYYPYTFGQGTKVEIKR

SEQ ID NO: 14 - protein, 108 aa, Homo sapiens
DIQMTQSPSSLSASVGDRVTITCRASQDIYLNLDWYQQKPGKAPKLLIKFGSELQSGVPSRFSGSGYGTDFTLTISNLQPEDFATYYCQPSFYFPYTFGQGTKVEIKW

SEQ ID NO: 15 - protein, 119 aa, Homo sapiens
EVQLLESGGGLVQPGGSLRLSCAASGFTFAHETMVWVRQAPGKGLEWVSHIPPVGQDPFYADSVKGRFTISRDDSKNTLYLQMNSLRAEDTAVYYCALLPKRGPWFDYWGQGTLVTVSS

SEQ ID NO: 16 - protein, 119 aa, Homo sapiens
EVQLWESGGGLVQPGGSLRLSCAASGFTFAHETMVWVRQAPGKGLEWVSHIPPDGQDPFYADSVKGRFTISRDNSKNTLYLQMNSLRAEDTAVYYCALLPKRGPWIDYWGQGTLVTVSS

SEQ ID NO: 17 - protein, 119 aa, Homo sapiens
EVQLSESGGGLVQPGGSLRLSCAASGFTFAHETMVWVRQAPGKGLEWVSHIPPDGQDPFYADSVKGRFTISRDNSKNTLYLQMNSLRAEDTAVYYCALLPNRGPWFDYWGQGTLVTVSS

SEQ ID NO: 18 - protein, 119 aa, Homo sapiens
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYYCALLPKRGPWFDYWGQGTQVTVSS

SEQ ID NO: 19 - protein, 116 aa, Homo sapiens
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 20 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 21 - protein, 116 aa, Homo sapiens
EVQLLESGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 22 - protein, 116 aa, Homo sapiens
EVQLLESGGGLVQPGGSLRLSCAASGFTFTAYPIMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 23 - protein, 116 aa, Homo sapiens
EVQLLESGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKIDYWGQGTLVTVSS

SEQ ID NO: 24 - protein, 116 aa, Homo sapiens
EVQLLESGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 25 - protein, 116 aa, Homo sapiens
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKSDYWGQGTLVTVSS

SEQ ID NO: 26 - protein, 116 aa, Homo sapiens
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKIDYWGQGTLVTVSS

SEQ ID NO: 27 - protein, 343 aa, Artificial
  Fusion
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK